(19) United States
(12) Patent Application Publication
(10) Pub. No.: US 2006/0135589 A1
(43) Pub. Date: Jun. 22, 2006

(54) 1h-Indazole-3-carboxamide compounds as cyclin dependent kinase (cdk) inhibitors

(75) Inventors: Valerio Berdino, Cambridge (GB); Alessandro Padova, Sovicille (IT); Paul Graham Wyatt, Cambridge (GB); Gordon Saxty, Cambridge (GB); Alison Jo-Anne Woolford, Cambridge (GB)

Correspondence Address: HESLIN ROTHENBERG FARLEY & MESITI PC, 5 COLUMBIA CIRCLE, ALBANY, NY, 12203, US

(73) Assignee: Astex Technology, Inc., Cambridge (GB)

(21) Appl. No.: 10/524,784

(22) PCT Filed: Aug. 8, 2003
(86) PCT No.: PCT/GB03/03491

§ 371 Date: Feb. 1, 2006

(30) Foreign Application Priority Data
Aug. 10, 2002 (GB) 0218625.2

(51) Int. Cl. A61K 31/416

(52) U.S. Cl. 514/406

(57) ABSTRACT

The invention provides a compound of the formula (I) for use in the prophylaxis or treatment of a disease state or condition mediated by a cyclin dependent kinase: 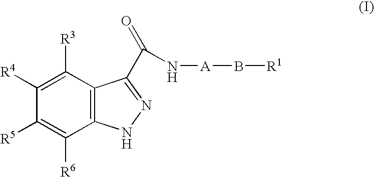 wherein
A is a group R² or CH₂—R² where R² is a carbocyclic or heterocyclic group having from 3 to 12 ring members; B is a bond or an acyclic linker group having a linking chain length of up to 3 atoms selected from C, N, S and O;
R¹ is hydrogen or a group selected from SO₂R^{b}, SO₂NR⁷R⁸, CONR⁷R⁸, NR⁷R⁹ and carbocyclic and heterocyclic groups having from 3 to 7 ring members;
R³, R⁴, R⁵ and R⁶ are the same or different and are each selected from hydrogen, halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is hydrogen or C₁₋₄ hydrocarbyl;
X¹ is O, S or NR^{c} and X² is ═O, ═S or ═NR^{c};
R⁷ is selected from hydrogen and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R⁸ is selected from R⁷ and carbocyclic and heterocyclic groups having from 3 to 12 ring members;
R⁹ is selected from R⁸, COR⁸ and SO₂R⁸;
or NR⁷R⁸ or NR⁷R⁹ may each form a heterocyclic group having from 5 to 12 ring members; but excluding the compounds N-[(morpholin-4-yl)phenyl-1H-indazole-3-carboxamide and N-[4-(acetylaminosulphonyl)phenyl-1H-indazole-3-carboxamide.

This invention relates to 3-substituted indazole compounds that inhibit or modulate the activity of cyclin dependent kinases (CDK), to the use of the compounds in the treatment or prophylaxis of disease states or conditions mediated by cyclin dependent kinases, and to novel compounds having cyclin dependent kinase inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, disease and conditions of the immune system, disease and conditions of the central nervous system, and angiogenesis.

The process of eukaryotic cell division may be broadly divided into a series of sequential phases termed G1, S, G2 and M. Correct progression through the various phases of the cell cycle has been shown to be critically dependent upon the spatial and temporal regulation of a family of proteins known as cyclin dependent kinases (CDKs) and a diverse set of their cognate protein partners termed cyclins. CDKs are cdc2 (also known as CDK1) homologous serine-threonine kinase proteins that are able to utilise ATP as a substrate in the phosphorylation of diverse polypeptides in a sequence dependent context. Cyclins are a family of proteins characterised by a homology region, containing approximately 100 amino acids, termed the “cyclin box” which is used in binding to, and defining selectivity for, specific CDK partner proteins.

Modulation of the expression levels, degradation rates, and activation levels of various CDKs and cyclins throughout the cell cycle leads to the cyclical formation of a series of CDK/cyclin complexes, in which the CDKs are enzymatically active. The formation of these complexes controls passage through discrete cell cycle checkpoints and thereby enables the process of cell division to continue. Failure to satisfy the pre-requisite biochemical criteria at a given cell cycle checkpoint, i.e. failure to form a required CDK/cyclin complex, can lead to cell cycle arrest and/or cellular apoptosis. Aberrant cellular proliferation, as manifested in cancer, can often be attributed to loss of correct cell cycle control. Inhibition of CDK enzymatic activity therefore provides a means by which abnormally dividing cells can have their division arrested and/or be killed. The diversity of CDKs, and CDK complexes, and their critical roles in mediating the cell cycle, provides a broad spectrum of potential therapeutic targets selected on the basis of a defined biochemical rationale.

Progression from the G1 phase to the S phase of the cell cycle is primarily regulated by CDK2, CDK3, CDK4 and CDK6 via association with members of the D and E type cyclins. The D-type cyclins appear instrumental in enabling passage beyond the G1 restriction point, where as the CDK2/cyclin E complex is key to the transition from the G1 to S phase. Subsequent progression through S phase and entry into G2 is thought to require the CDK2/cyclin A complex. Both mitosis, and the G2 to M phase transition which triggers it, are regulated by complexes of CDK1 and the A and B type cyclins.

During G1 phase Retinoblastoma protein (Rb), and related pocket proteins such as p130, are substrates for CDK(2, 4, & 6)/cyclin complexes. Progression through G1 is in part facilitated by hyperphosphorylation, and thus inactivation, of Rb and p130 by the CDK(4/6)/cyclin-D complexes. Hyperphosphorylation of Rb and p 130 causes the release of transcription factors, such as E2F, and thus the expression of genes necessary for progression through G1 and for entry into S-phase, such as the gene for cyclin E. Expression of cyclin E facilitates formation of the CDK2/cyclin E complex which amplifies, or maintains, E2F levels via further phosphorylation of Rb. The CDK2/cyclin E complex also phosphorylates other proteins necessary for DNA replication, such as NPAT, which has been implicated in histone biosynthesis. G1 progression and the G1/S transition are also regulated via the mitogen stimulated Myc pathway, which feeds into the CDK2/cyclin E pathway. CDK2 is also connected to the p53 mediated DNA damage response pathway via p53 regulation of p21 levels. p21 is a protein inhibitor of CDK2/cyclin E and is thus capable of blocking, or delaying, the G1/S transition. The CDK2/cyclin E complex may thus represent a point at which biochemical stimuli from the Rb, Myc and p53 pathways are to some degree integrated. CDK2 and/or the CDK2/cyclin E complex therefore represent good targets for therapeutics designed at arresting, or recovering control of, the cell cycle in aberrantly dividing cells.

The exact role of CDK3 in the cell cycle is not clear. As yet no cognate cyclin partner has been identified, but a dominant negative form of CDK3 delayed cells in G1, thereby suggesting that CDK3 has a role in regulating the G1/S transition.

Although most CDKs have been implicated in regulation of the cell cycle there is evidence that certain members of the CDK family are involved in other biochemical processes. This is exemplified by CDK5 which is necessary for correct neuronal development and which has also been implicated in the phosphorylation of several neuronal proteins such as Tau, NUDE-1, synapsin1, DARPP32 and the Munc18/Syntaxin1A complex. Neuronal CDK5 is conventionally activated by binding to the p35/p39 proteins. CDK5 activity can, however, be deregulated by the binding of p25, a truncated version of p35. Conversion of p35 to p25, and subsequent deregulation of CDK5 activity, can be induced by ischemia, excitotoxicity, and β-amyloid peptide. Consequently p25 has been implicated in the pathogenesis of neurodegenerative diseases, such as Alzheimer's, and is therefore of interest as a target for therapeutics directed against these diseases.

CDK7 is a nuclear protein that has cdc2 CAK activity and binds to cyclin H. CDK7 has been identified as component of the TFIIH transcriptional complex which has RNA polymerase II C-terminal domain (CTD) activity. This has been associated with the regulation of HIV-1 transcription via a Tat-mediated biochemical pathway. CDK8 binds cyclin C and has been implicated in the phosphorylation of the CTD of RNA polymerase II. Similarly the CDK9/cyclin-T1 complex (P-TEFb complex) has been implicated in elongation control of RNA polymerase II. PTEF-b is also required for activation of transcription of the HIV-1 genome by the viral transactivator Tat through its interaction with cyclin T1. CDK7, CDK8, CDK9 and the P-TEFb complex are therefore potential targets for anti-viral therapeutics.

At a molecular level mediation of CDK/cyclin complex activity requires a series of stimulatory and inhibitory phosphorylation, or dephosphorylation, events. CDK phosphorylation is performed by a group of CDK activating kinases (CAKs) and/or kinases such as wee1, Myt1 and Mik1. Dephosphorylation is performed by phosphatases such as cdc25(a & c), pp2a, or KAP.

CDK/cyclin complex activity may be further regulated by two families of endogenous cellular proteinaceous inhibitors: the Kip/Cip family, or the INK family. The INK proteins specifically bind CDK4 and CDK6. p16^{Ink4} (also known as MTS1) is a potential tumour suppressor gene that is mutated, or deleted, in a large number of primary cancers. The Kip/Cip family contains proteins such as p21^{Cip1,Waf1}, p27^{Kip1} and p57^{Kip2}. As discussed previously p21 is induced by p53 and is able to inactivate the CDK2/cyclin(E/A) and CDK4/cyclin(D1/D2/D3) complexes. Atypically low levels of p27 expression have been observed in breast, colon and prostate cancers. Conversely over expression of cyclin E in solid tumours has been shown to correlate with poor patient prognosis. Over expression of cyclin D1 has been associated with oesophageal, breast, squamous, and non-small cell lung carcinomas.

The pivotal roles of CDKs, and their associated proteins, in co-ordinating and driving the cell cycle in proliferating cells have been outlined above. Some of the biochemical pathways in which CDKs play a key role have also been described. The development of monotherapies for the treatment of proliferative disorders, such as cancers, using therapeutics targeted generically at CDKs, or at specific CDKs, is therefore potentially highly desirable. CDK inhibitors could conceivably also be used to treat other conditions such as viral infections, autoimmune diseases and neuro-degenerative diseases, amongst others. CDK targeted therapeutics may also provide clinical benefits in the treatment of the previously described diseases when used in combination therapy with either existing, or new, therapeutic agents. CDK targeted anticancer therapies could potentially have advantages over many current antitumour agents as they would not directly interact with DNA and should therefore reduce the risk of secondary tumour development.

WO 02/34721 from Du Pont discloses a class of indeno [1,2-c]pyrazol-4-ones as inhibitors of cyclin dependent kinases.

WO 01/81348 from Bristol Myers Squibb describes the use of 5-thio-, sulphinyl- and sulphonylpyrazolo[3,4-b]-pyridines as cyclin dependent kinase inhibitors.

WO 00/62778 also from Bristol Myers Squibb discloses a class of protein tyrosine kinase inhibitors.

WO 01/72745A1 from Cyclacel describes 2-substituted 4-heteroaryl-pyrimidines and their preparation, pharmaceutical compositions containing them and their use as inhibitors of cyclin-dependant kinases (CDKs) and hence their use in the treatment of proliferative disorders such as cancer, leukaemia, psoriasis and the like.

WO 99/21845 from Agouron describes 4-aminothiazole derivatives for inhibiting cyclin-dependent kinases (CDKs), such as CDK1, CDK2, CDK4, and CDK6. The invention is also directed to the therapeutic or prophylactic use of pharmaceutical compositions containing such compounds and to methods of treating malignancies and other disorders by administering effective amounts of such compounds.

WO 01/53274 from Agouron discloses as CDK kinase inhibitors a class of compounds which can comprise an amide-substituted benzene ring linked to an N-containing heterocyclic group. Although indazole compounds are not mentioned generically, one of the exemplified compounds comprises an indazole 3-carboxylic acid anilide moiety linked via a methylsulphanyl group to a pyrazolopyrimidine.

WO 01/98290 (Pharmacia & Upjohn) discloses a class of 3-aminocarbonyl-2-carboxamido thiophene derivatives as protein kinase inhibitors. The compounds are stated to have multiple protein kinase activity.

GB 1301882, U.S. Pat. No. 3,705,175, DE 2,135,398 (all to Egyt), and Ferenc et al., Magyar Kemikusok Lapja, 1975, 30(4), 208-215, each disclose 6,7-dimethoxyindazole-3-carboxylic acid amides as anti-inflammatory and analgesic agents.

U.S. Pat. No. 3,457,269 and U.S. Pat. No. 3,145,215 (both to Sterling Drug) disclose indazole-3-carboxylic acid amides, including anilides, cycloaliphatic amides and pyridylamides, as hypotensive agents.

WO 01/53268 and WO 01/02369 from Agouron disclose compounds that mediate or inhibit cell proliferation through the inhibition of protein kinases such as cyclin dependent kinase or tyrosine kinase. The Agouron compounds have an aryl or heteroaryl ring attached directly or though a CH═CH or CH═N group to the 3-position of an indazole ring.

WO 02/10137 (Signal Pharmaceuticals) discloses a class of indazole derivatives as selective inhibitors of JNK kinase. The indazole derivatives have an aryl, heteroaryl or heterocyclic group linked to the indazole 3-position through an akylene or alkenylene group.

U.S. Pat. No. 6,340,685 (Scios) discloses a class of bicyclic heterocyclic compounds as selective P38 MAP kinase inhibitors. Indazoles are not specifically disclosed.

WO 02/24635 (Fujisawa) discloses a class of amino alcohol derivatives as α-3 adrenergic receptor agonists. The compounds can contain an indazole 3-carboxylic acid anilide group linked to the amino alcohol group.

JP 04089489 (Nisshin), JP 03223280 (Dainippon), JP 05230057 (Dainippon), JP 04005289 (Hokuriku), JP 06135960 (Dainippon), EP 0499995 (Nisshin), EP 0623621 (Nisshin), WO 96/38420 (Nisshin), EP 0708105 (Nisshin), EP 0358903 (Dainippon), Harada et al. Chem. Pharm. Bull., 43 (11), 1912-1930 (1995), Harada et al. Chem. Pharm. Bull., 44 (12), 2205-2212 (1996) and Morie et al. Synthetic Communications, 27(4), 559-566 (1997) each disclose indazole 3-carboxamides in which the amide nitrogen is linked to a non-aromatic cyclic amino group. The compounds are described as being active as 5-HT receptor modulators.

EP 0410509 (Duphar) discloses, as 5-HT receptor antagonists, a class of indazole 3-carboxamides in which the amide nitrogen is linked to an imidazolylmethyl group.

Indazole carboxylic acid derivatives are also disclosed as 5-HT receptor modulators in WO 93/03725 (SmithKline Beecham), EP 0261964 (Beecham), EP 0517984 (Merrell Dow), U.S. Pat. No. 5,654,320 (Eli Lilly), EP 0908452 (Eli Lilly), EP 0908459 (Eli Lilly) and EP 0732333 (Eli Lilly).

U.S. Pat. No. 5,190,953 (A. H. Robins) describes a class of azabicyclic compounds that can contain an indazole group and which are stated to increase gastric motility.

U.S. Pat. No. 5,273,972 (A. H. Robins), U.S. Pat. No. 5,318,977 (Searle), WO 00/63215 (Sanofi-Synthelabo), WO 02/32416 (Depomed), WO 95/27490 (Sandoz), DE 3827253 (Sandoz), WO 91/09593 (Beecham), WO 92/05174 (Beecham), WO 93/07147 (SmithKline Beecham), WO 94/10174 (SmithKline Beecham), WO 96/02537 (SmithKline Beecham) and EP 0200444 (Beecham) also disclose classes of fused bicyclic heterocyclic compounds as 5-HT receptor modulators.

WO 01/58869 (Bristol Myers Squibb) discloses a number of indazole-3-carboxamide derivatives as cannabinoid receptor antagonists.

WO 02/20484 (Astra Zeneca) discloses a broad class of compounds, including compounds containing an indazole group, as modulators of chemokine receptor activity. No indazoles are exemplified however.

WO 02/053534 (Daichii) discloses a class of carboxylic acids and their esters as VLA inhibitors. The compounds, which are stated to be useful in the treatment of various disease states including inflammatory conditions, can comprise a halogenated phenyl acetic acid moiety linked to an indazole-3-carboxamido group.

WO 93/01169 (Merck) describes a class of compounds that have tachykinin receptor antagonist activity. The compounds may contain an indazole group, but there are no examples of indazole-3-carboxamides.

WO 98/03494 (Neurogen) discloses a class of 1-phenyl-1-piperazino-cycloalkanes and aza-cycloalkanes in which the phenyl group can form part of an indazole-3-carboxylic acid phenylamide. The compounds are disclosed as being capable of binding to mammalian neuropeptide Y1.

WO 99/29661 (Astra) describes a broad class of adamantane derivatives and oxa-adamantane derivatives as being useful in the treatment of rheumatoid arthritis, osteoarthritis, psoriasis and the growth and metastasis of malignant cells. However, there are no examples of indazoles.

WO 01/57024 (University College) discloses the use of various compounds, including indazoles, for blocking voltage dependent sodium channels.

WO 01/83472 (Acadia) describes a class of bicyclic heteroaryl compounds as muscarinic agonists. One of the exemplified compounds is the 1-butyl-4-piperidinomethyl amide of indazole-3-carboxylic acid.

EP 01013276 (Pfizer) discloses a class of compounds as modulators of chemokine activity that can be used in the treatment of inflammatory conditions. Indazoles are amongst the large list of compounds mentioned but there are no examples of indazoles.

WO 02/16318 (Pacific Corporation) discloses vanilloid receptor modulators for the treatment of inflammatory diseases. The modulator compounds can be indazoles but there is no disclosure of indazole-3-carboxamides.

WO 02/059112 (Vertex) discloses pyrazoles as protein kinase inhibitors but there are no examples of indazole-3-carboxamides.

WO 99/49856 (Genentech) discloses compounds that are useful in treating CD11/CD18 adhesion receptor mediated disorders such as inflammation, psoriasis and rheumatoid arthritis. The compounds can contain an indazole unit but there are no examples of indazole-3-carboxamides.

JP 01117882 (Dainippon) discloses heteroarylamides for use in treating certain disorders of the gastrointestinal system.

JP 11130750 (Fujisawa) discloses a class of arylamides for use in the treatment of CNS disorders.

WO 00/18738 (Zeneca) discloses a class of bis-amidophenyl compounds that act as inhibitors of cytokine production and which are stated to be useful in the treatment of inflammatory and allergic disease states. The compounds can contain an indazole unit but there are no examples of indazoles.

Kaneko et al. Nippon Shashin Gakkaishi 1995, 58(2), 122-8 discloses the use of indazole-3-carboxylic acid phenylamide as a cyan dye forming compound.

Duykina et al., ZH. Obsh. Khim. 32, 81-84 (1962) discloses various indazole derivatives, including indazole-3-carboxylic acid 4-methylbenzylamide.

Hannig et al. Pharmazie, 28, 11/12, 720-723 (1973) describes a number of 5-methylindazole-3-carboxylic acid phenylamides and benzylamides as anti-inflammatory agents.

Schaus et al., J. Med. Chem., 41, 1943-1955 (1998) disclose a number of indazole-3-carboxamides as 5-HT₄ receptor antagonists.

Nagarajan et al., Proc. Indian Acad. Sci., 86A, 25-39 (1977) describes the synthesis of indazole-3-carboxylic acid methoxyphenylamide.

Peter et al., Acta Pharmaceutica Hungarica, 43, 147-151 (1973) describes the preparation of a class of indazole-3-carboxylic acid phenylalkylamides.

### SUMMARY OF THE INVENTION

The invention provides compounds that have cyclin dependent kinase inhibiting or modulating activity, and which it is envisaged will be useful in preventing or treating disease states or conditions mediated by the cyclin dependent kinases.

Accordingly, in one aspect, the invention provides a compound of the formula (I) as defined herein for use in the prophylaxis or treatment of a disease state or condition mediated by a cyclin dependent kinase.

The invention also provides the use of a compound of the formula (I) as defined herein for the manufacture of a medicament for the prophylaxis or treatment of a disease state or condition mediated by a cyclin dependent kinase.

In a further aspect, the invention provides a method for the prophylaxis or treatment of a disease state or condition mediated by a cyclin dependent kinase, which method comprises administering to a subject in need thereof a compound of the formula (I) as defined herein.

This invention also provides a method for treating a disease or condition comprising or arising from abnormal cell growth in a mammal, which method comprises administering to the mammal a compound of the formula (I) as defined herein in an amount effective in inhibiting abnormal cell growth.

This invention further provides a method for treating a disease or condition comprising or arising from abnormal cell growth in a mammal, the method comprising administering to the mammal a compound of the formula (I) as defined herein in an amount effective to inhibit CDK2 activity.

In another aspect, the invention provides a method of inhibiting a cyclin dependent kinase, which method comprises contacting the kinase with a kinase-inhibiting compound of the formula (I) as defined herein.

The invention further provides a method of modulating a cellular process (for example cell division) by inhibiting the activity of a cyclin dependent kinase using a compound of the formula (I) as defined herein.

The compounds of the invention are represented by the general formula (I): 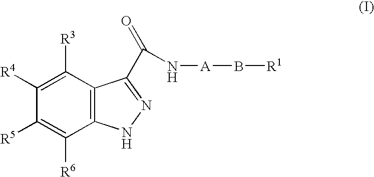
wherein

A is a group R² or CH₂—R² where R² is a carbocyclic or heterocyclic group having from 3 to 12 ring members;

B is a bond or an acyclic linker group having a linking chain length of up to 3 atoms selected from C, N, S and O;

R¹ is hydrogen or a group selected from SO₂R^{b}, SO₂NR⁷R⁸, CONR⁷R⁸, NR⁷R⁹ and carbocyclic and heterocyclic groups having from 3 to 7 ring members;

R³, R⁴, R⁵ and R⁶ are the same or different and are each selected from hydrogen, halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is hydrogen or C₁₋₄ hydrocarbyl;

X¹ is O, S or NR^{c} and X² is ═O, ═S or ═NR^{c};

R⁷ is selected from hydrogen and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R⁸ is selected from R⁷ and carbocyclic and heterocyclic groups having from 3 to 12 ring members;

R⁹ is selected from R⁸, COR⁸ and SO₂R⁸;

or NR⁷R⁸ or NR⁷R⁹ may each form a heterocyclic group having from 5 to 12 ring members;

but excluding the compounds N-[(morpholin-4-yl)phenyl-1H-indazole-3-carboxamide and N-[4-(acetylaminosulphonyl)phenyl-1H-indazole-3-carboxamide.

The group A is a group R² or CH₂—R² where R² is a carbocyclic or heterocyclic group having from 3 to 12 ring members. In one particular embodiment, A is a group R².

References to “carbocyclic” and “heterocyclic” groups as used herein, either with regard to the group R² or any other substituent group, unless the context indicates otherwise include both aromatic and non-aromatic ring systems. Thus, for example, the term “carbocyclic and heterocyclic groups having from 3 to 12 ring members” includes within its scope aromatic, non-aromatic, unsaturated, partially saturated and fully saturated carbocyclic and heterocyclic ring systems.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. The aryl or heteroaryl groups can be monocyclic or bicyclic groups and can be unsubstituted or substituted with one or more substituents, for example one or more groups R¹⁰ as defined below.

Examples of heteroaryl groups are monocyclic and bicyclic groups containing from five to twelve ring members, and more usually from five to ten ring members.

The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of a pyrazole, imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl groups include but are not limited to pyridyl, pyrrolyl, furanyl, thiophenyl, imidazolyl, oxazolyl, oxadiazolyl, oxatriazolyl, isoxazolyl, thiazolyl, isothiazolyl, pyrazolyl, pyrazinyl, pyrimidinyl, pyridazinyl, triazinyl, triazolyl, tetrazolyl, quinolinyl, isoquinolinyl, benzfuranyl, benzthiophenyl, chromanyl, thiochromanyl, benzimidazolyl, benzoxazolyl, benzisoxazole, benzthiazolyl and benzisothiazole, isobenzofuranyl, isoindolyl, indolizinyl, indolinyl, isoindolinyl, purinyl (e.g., adenine, guanine), indazolyl, benzodioxolyl, chromenyl, isochromenyl, chroman, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl.

In the context of the group R², one particular sub-group of compounds of the formula (I) is the group wherein R² is selected from pyridyl, quinolinyl, isoquinolinyl and thiadiazolyl.

The pyridyl group can be a 2-pyridyl, 3-pyridyl or 4-pyridyl group but preferably it is a 3-pyridyl group.

Examples of carbocyclic aryl groups include phenyl, naphthyl, indenyl, and tetrahydronaphthyl.

In the context of the group R², preferred aryl groups are groups based on a benzene ring. Thus it may be, for example, a phenyl group which has no substituents other than the group B, or has one or more further substituents R¹⁰ as defined herein.

Examples of non-aromatic heterocyclic groups are groups having from 3 to 12 ring members, more usually 5 to 10 ring members. Such groups can be monocyclic or bicyclic, for example, and typically have from 1 to 5 heteroatom ring members (more usually 1, 2, 3 or 4 heteroatom ring members), usually selected from nitrogen, oxygen and sulphur. The heterocylic groups can contain, for example, cyclic ether moieties (e.g as in tetrahydrofuran and dioxane), cyclic thioether moieties (e.g. as in tetrahydrothiophene), cyclic amine moieties (e.g. as in pyrrolidine), cyclic amides (such as a pyrrolidinone, piperidone or caprolactam), cyclic sulphonamides (such as an isothiazolidine 1,1-dioxide, [1,2]thiazinane 1,1-dioxide or [1,2]thiazepane 1,1-dioxide), cyclic sulphones (e.g. as in sulpholane and sulpholene)), cyclic sulphoxides, and combinations thereof.

Particular examples include morpholine, piperidine, pyrrolidine, pyrrolidone, tetrahydrofuran, tetrahydrothiophene, dioxan, tetrahydropyran, imidazoline, imidazolidinone, oxazoline, thiazoline, piperazine, and N-alkyl piperazines such as N-methyl piperazine. In general, preferred non-aromatic heterocyclic groups include tetrahydrofuran, morpholine, piperazine, piperidine, pyrrolidine and pyrrolidone.

The carbocyclic and heterocyclic groups can be polycyclic fused ring systems but it is preferred that they are not bridged ring systems such as bicycloalkanes, tricycloalkanes and their oxa- and aza analogues (e.g. adamantane and oxa-adamantane). For an explanation of the distinction between fused and bridged ring systems, see Advanced Organic Chemistry, by Jerry March, 4^{th} Edition, Wiley Interscience, pages 131-133, 1992.

The carbocyclic and heterocyclic groups can each be unsubstituted or substituted by one or more substituent groups R¹⁰ in addition to the group B—R¹. For example, the carbocyclic and heterocyclic groups can be unsubstituted or substituted by 1, 2, 3 or 4 substituents. Where the carbocyclic or heterocyclic group is monocyclic or bicyclic, typically it is unsubstituted or has 1, 2 or 3 substituents, preferably 0, 1 or 2 substituents, and more preferably 0 or 1 substituent. In one embodiment, the carbocyclic and heterocyclic groups have no substituents in addition to the group B—R¹.

The group R¹⁰ is selected from halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is hydrogen or C₁₋₄ hydrocarbyl;

X¹ is O, S or NR^{c} and X² is ═O, ═S or ═NR^{c}.

Where the substituent group R¹⁰ comprises or includes a carbocyclic or heterocyclic group, the said carbocyclic or heterocyclic group may be unsubstituted or may itself be substituted with one or more further substituent groups R¹⁰. In one sub-group of compounds of the formula (I), such further substituent groups R¹⁰ may include carbocyclic or heterocyclic groups. In another sub-group of compounds of the formula (I), the said further substituents do not include carbocyclic or heterocyclic groups but are otherwise selected from the groups listed above in the definition of R¹⁰.

In one general embodiment, the substituent groups R¹⁰ may be selected from halogen, hydroxy, trifluoromethyl, cyano, nitro, amino; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen and a C₁₋₈ hydrocarbyl group optionally substituted by one or more substituents selected from hydroxy, oxo, halogen, cyano, nitro, amino, mono- or di-C₁₋₄ hydrocarbylamino and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is hydrogen or C₁₋₄ hydrocarbyl;

X¹ is O, S or NR^{c} and X² is ═O, ═S or ═NR^{c}.

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone, except where otherwise stated. Examples of such groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or substituted by one or more substituents as defined herein. The examples and preferences expressed below apply to each of the hydrocarbyl substituent groups or hydrocarbyl-containing substituent groups referred to in the various definitions of substituents for compounds of the formula (I) unless the context indicates otherwise.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

When present, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituent can be a partially fluorinated or perfluorinated group such as trifluoromethyl. In one embodiment preferred substituents include monocyclic carbocyclic and heterocyclic groups having 3-7 ring members.

One or more carbon atoms of a hydrocarbyl group may optionally be replaced by O, S, SO, SO₂, NR^{c}, X¹C(X²), C(X²)X¹ or X¹C(X²)X¹ wherein X¹ and X² are as hereinbefore defined. For example, 1, 2, 3 or 4 carbon atoms of the hydrocarbyl group may be replaced by one of the atoms or groups listed, and the replacing atoms or groups may be the same or different. Examples of groups in which a carbon atom of the hydrocarbyl group has been replaced by a replacement atom or group as defined above include ethers and thioethers (C replaced by O or S), amides, esters, thioamides and thioesters (C replaced by X¹C(X²) or C(X²)X¹), sulphones and sulphoxides (C replaced by SO or SO₂) and amines (C replaced by NR^{c}).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members.

The definition “R^{a}—R^{b}” as used herein, either with regard to substituents present on the carbocyclic or heterocyclic moiety R², or with regard to other substituents present at other locations on the compounds of the formula (I), includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S, SO, SO₂ NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined.

Examples of hydrocarbyl, carbocyclic and heterocyclic groups are as set out above.

In one general embodiment, each substituent group R¹⁰, when present, is other than a carboxy group or a hydrocarbyl group terminated by a carboxy group or alkoxycarbonyl group.

In the compounds of the formula (I), B is a bond or an acyclic linker group. The linker group has a linking chain length of up to 3 atoms: in other words the number of atoms in the backbone of the linker group is 1, 2 or 3. Thus, for example, a group —CH₂— has a linking chain length of one, whilst a group —CH₂—CH₂— has a linking chain length of two.

It is preferred that B is a bond or a linker group having a linking chain length of 1 atom.

The atoms making up the backbone of the linker group are selected from C, N, S and O, but preferably the atoms defining the linking chain length are all carbon atoms.

The linker group is typically a straight chain group. By “straight chain” is meant a group that has no branched side chains. In general a straight chain linker group may bear single atom substituents such as halogen and oxo, or substituents each of 1, 2 or 3 atoms, but would not usually have hydrocarbon substituents such as methyl, or larger multi-atom substituents each having four atoms or more such as methoxy or trifluoromethyl for example.

A preferred linker group B is a group (CH₂)ⁿ wherein n is 1, 2 or 3, more preferably 1 or 2, and most preferably 1.

The groups R³, R⁴, R⁵ and R⁶ are the same or different and are each selected from hydrogen, halogen, hydroxy, trifluoromethyl, cyano, nitro, carboxy, amino, carbocyclic and heterocyclic groups having from 3 to 7 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 (typically 3 to 10 and more usually 5 to 10) ring members, and a C₁₋₈ hydrocarbyl group optionally substituted by one or more substituents selected from hydroxy, oxo, halogen, cyano, nitro, amino, mono- or di-C₁₋₄ hydrocarbylamino, monocyclic carbocyclic and heterocyclic groups having from 3 to 12 (typically 3 to 10 and more usually 5 to 10) ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is hydrogen or C₁₋₄ hydrocarbyl; and

X¹ is O, S or NR^{c} and X² is ═O, ═S or ═NR^{c}.

It is preferred that R³ is hydrogen or a group selected from halogen, hydroxy, cyano, trifluoromethyl, amino and R^{a}—R^{b}.

More preferably R³ is hydrogen, C₁₋₆ alkyl, fluorine or chlorine, and most preferably R³ is hydrogen.

It is also preferred that R⁵ is hydrogen or a group selected from halogen, hydroxy, cyano, trifluoromethyl, amino and R^{a}—R^{b}.

More preferably R⁵ is hydrogen, C₁₋₆ alkyl, fluorine or chlorine, and most preferably R⁵ is hydrogen.

In one particular embodiment, R³ and R⁵ are both hydrogen.

It is preferred that R⁴ is selected from hydrogen, halogen, hydroxy, trifluoromethyl, cyano, amino, carbocyclic and heterocyclic groups having from 3 to 12 ring members (typically 3 to 10 and more usually 5 to 10 ring members), and a group R^{a}—R^{b}.

More preferably, R⁴ is selected from hydrogen, halogen, a heterocyclic group and a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 5 to 10 ring members, and a C₁₋₈ hydrocarbyl group optionally substituted by one or more substituents selected from hydroxy, oxo, halogen, cyano, nitro, amino, mono- or di-C₁₋₄ hydrocarbylamino, monocyclic carbocyclic and heterocyclic groups having from 5 to 10 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹.

Within the above definition of preferred groups R⁴, one particular group of compounds is the group in which R⁴ is selected from hydrogen, halogen, a heterocyclic group, a group O-Het where Het is a heterocyclic group having from 5 to 10 ring members, C₁₋₆ alkyl, C₁₋₆ alkoxy, C(O)NR^{c}R^{b} and SO₂NR^{c}R^{b} wherein R^{b} is hydrogen or C₁₋₆ alkyl.

R⁶ is preferably selected from hydrogen, methyl, amino, fluorine and chlorine, and more preferably hydrogen and amino. Most preferably, R⁶ is hydrogen.

In one particular group of compounds of the formula (I), R³, R⁵ and R⁶ each are hydrogen.

In one general embodiment of the invention, the compounds of the formula (I) may be such that when R¹ is SO₂NR⁷R⁸, neither of R⁷ and R⁸ is a C₁₋₈ hydrocarbyl group in which the carbon atom attached to the nitrogen atom of the group SO₂NR⁷R⁸ is substituted by an oxo group.

In another general embodiment, the compounds of the formula (I) may be such that R¹ is other than the heterocyclic group N-morpholino when B is a bond and A is R² wherein R² is aryl.

Novel Compounds

Many of the compounds of the formula (I) are novel. Accordingly, in another aspect, the invention provides a compound of the formula (II): 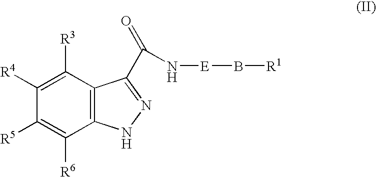
wherein

E is a group R¹² or CH₂—R^{12a} where R¹² is a substituted or unsubstituted, non-bridged, carbocyclic or heterocyclic group having from 3 to 12 ring members, other than a diazacycloalkyl moiety, and R^{12a} is an unsubstituted or substituted aryl or heteroaryl group having from 5 to 12 ring members;

B is a bond or an acyclic linker group having a linking chain length of up to 3 atoms selected from C, N, S and O;

R¹ is hydrogen or a group selected from SO₂R^{b}, SO₂NR⁷R⁸, CONR⁷R⁸, NR⁷R⁹ and carbocyclic and heterocyclic groups having from 3 to 7 ring members;

R³, R⁴, R⁵ and R⁶ are the same or different and are each selected from hydrogen, halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is hydrogen or C₁₋₄ hydrocarbyl;

X¹ is O, S or NR^{c} and X² is ═O, ═S or ═NR^{c};

R⁷ is selected from hydrogen and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R⁸ is selected from R⁷ and carbocyclic and heterocyclic groups having from 3 to 12 ring members;

R⁹ is selected from R⁸, COR⁸ and SO₂R⁸;

or NR⁷R⁸ or NR⁷R⁹ may each form a heterocyclic group having from 5 to 12 ring members;

and the optional substituents for the groups R¹² and R¹²a can be one or more substituent groups R¹⁰ selected from halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is hydrogen or C₁₋₄ hydrocarbyl;

X¹ is O, S or NR^{c} and X² is ═O, ═S or ═NR^{c}; with the provisos that:

(a) when R¹² is an azacycloalkyl or diazacycloalkyl group, at least one nitrogen atom of the azacycloalkyl or diazacycloalkyl group is substituted by an acyl, sulphinyl or sulphonyl group;

(b) when E is a substituted phenyl group, the or each substituent is other than a 5-7 membered non-aromatic ring (such as cyclohexyl) having attached thereto a diazacycloalkyl moiety (such as piperazine), a nitrogen atom of which moiety bears an aryl or heteroaryl substituent; and

(c) R¹² and R^{12a} are each other than a substituted or unsubstituted imidazole moiety;

but excluding the following:

(i) N-[(morpholin-4-yl)phenyl-1H-indazole-3-carboxamide;

(ii) N-[4-(acetylaminosulphonyl)phenyl-1H-indazole-3-carboxamide;

(iii) compounds wherein E is phenyl, R¹ is NR⁷R⁹ and B is a group —CH(CH₂OH)CH₂—;

(iv) compounds wherein R³ and R⁶ are both hydrogen and R⁴ and R⁵ are both methoxy;

(v) compounds wherein R³ to R⁶ are all hydrogen, E is unsubstituted pyridyl or pyridylmethyl, B is a bond and R¹ is hydrogen;

(vi) compounds wherein E is phenyl substituted with one or more of alkyl, alkoxy, alkylsulphanyl, alkylsulphinyl other than meta-alkylsulphinyl, alkylsulphonyl other than meta-alkylsulphonyl, halogen, nitro and trihalomethyl, B is a bond, and R¹ is hydrogen;

(vii) compounds wherein E is a thiophene group bearing a 3-aminocarbonyl substituent;

(viii) the compound wherein E is unsubstituted phenyl or para-methoxyphenyl, and each of R³ to R⁶ is hydrogen;

(ix) N-4-methylbenzyl-1H-indazole-3-carboxamide;

(x) compounds wherein R³, R⁵ and R⁶ are each hydrogen, R⁴ is methyl and A is unsubstituted benzyl, unsubstituted phenyl, methylphenyl, meta-trifluoromethylphenyl, and ortho-methoxyphenyl;

(xi) compounds in which E is a 2,2-dimethyl-1,3-dioxane ring;

(xii) compounds containing a benzene ring substituted by a pair of meta-oriented carboxamido moieties;

(xiii) compounds wherein E is a trisubstituted phenyl group and two of the substitutents are fluoro and chloro respectively.

In one embodiment, E-B—R¹ may be other than a diazine or triazine substituted by a monocyclic pyrazolyl group or a bicyclic fused pyrazolyl group.

In another embodiment, E-B—R¹ may be other than a saturated azabicyclic moiety or an imidazolyl moiety.

In another general embodiment, the compound of the formula (II) is other than one in which E is unsubstituted pyridyl or pyridylmethyl, B is a bond and R¹ is hydrogen.

In a further embodiment, when E-B—R¹ is an unsubstituted phenyl group, R³ to R⁶ are other than a group R^{a}—R^{b} wherein R^{a} is a bond and R^{b} is a substituted C₃-C₈ hydrocarbyl group having two or more substituents, one of which contains an unsubstituted or substituted amino group.

The invention also provides a group of novel compounds of the formula (III): 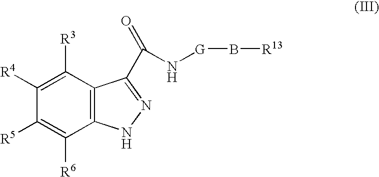
wherein

G is a group R¹⁴ or CH₂—R¹⁴ where R¹⁴ is a carbocyclic group having from 3 to 12 ring members;

B is a bond or an acyclic linker group having a linking chain length of up to 3 atoms selected from C, N, S and O;

R¹³ is a group selected from SO₂NR⁷R⁸, CONR⁷R⁸, NR⁷R⁹ and carbocyclic and heterocyclic groups having from 3 to 7 ring members;

R³, R⁴, R⁵ and R⁶ are the same or different and are each selected from hydrogen, halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is hydrogen or C₁₋₄ hydrocarbyl;

X¹ is O, S or NR^{c} and X² is ═O, ═S or ═NR^{c};

R⁷ is selected from hydrogen and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R⁸ is selected from R⁷ and carbocyclic and heterocyclic groups having from 3 to 12 ring members;

R⁹ is selected from R⁸, COR⁸ and SO₂R⁸;

or NR⁷R⁸ or NR⁷R⁹ may each form a heterocyclic group having from 5 to 12 ring members;

but excluding the compounds N-[(morpholin-4-yl)phenyl-1H-indazole-3-carboxamide and N-[4-(acetylaminosulphonyl)phenyl-1H-indazole-3-carboxamide; and further excluding;

(i) compounds wherein G is phenyl, R¹ is NR⁷R⁸ and B is a group —CH(CH₂OH)CH₂—;

(ii) compounds wherein R³ and R⁶ are both hydrogen and R⁴ and R⁵ are both methoxy.

One sub-group of novel compounds of the invention is represented by the general formula (IV): 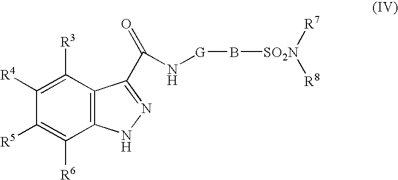
wherein R³ to R⁸, G and B are as hereinbefore defined.

Within the sub-group of compounds of the formula (IV), preferred compounds include those wherein G is a group R¹⁴ wherein R¹⁴ is an aryl group having six ring members and B is a bond or a methylene group.

Another preferred group of compounds within formula (IV) is the group of compounds in which R⁷ and R⁸ are selected from hydrogen and C₁₋₄ alkyl or R⁷ and R⁸ together with the nitrogen atom form a saturated five or six membered heterocyclic ring having one or two heteroatoms.

Examples of such compounds include compounds wherein R⁷ and R⁸ together with the nitrogen atom form a saturated heterocyclic ring selected from morpholino, piperidino, piperazino and pyrrolidino.

Further particular examples are compounds in which R⁷ is hydrogen and R⁸ is hydrogen or methyl.

Another group of novel compounds of the invention is represented by the general formula (V): 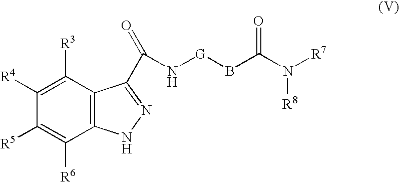
wherein R³ to R⁸, G and B are as hereinbefore defined.

Within the sub-group of compounds of the formula (V), preferred compounds include those wherein G is a group R¹⁴ wherein R¹⁴ is an aryl group having six ring members and B is a bond or a methylene group.

A further novel group of compounds of the invention is represented by the general formula (VI): 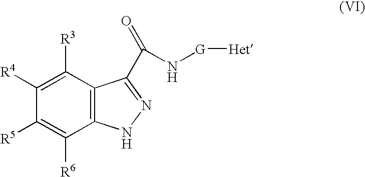
wherein R³ to R⁶ and G are as hereinbefore defined and Het′ is a heterocylic group having from 3 to 7 ring members, but excluding the compound N-[(morpholin-4-yl)phenyl]-1H-indazole-3-carboxamide.

Within the sub-group of compounds of the formula (VI), preferred compounds include those wherein G is a group R¹⁴ wherein R¹⁴ is an aryl group having six ring members and B is a bond or a methylene group.

In one sub-group of compounds of the formula (VI), a carbon atom of the heterocyclic group Het′ is linked to the group G.

The group Het′ can be, for example, a five membered heteroaryl ring containing 2 or more nitrogen ring members. Examples of such groups include tetrazolyl, pyrrolidonyl (e.g. N-pyrrolidonyl), oxazolyl and imidazolyl.

A further sub-group of novel compounds of the invention is represented by the formula (VII): 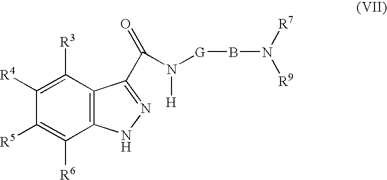
wherein R³ to R⁷, R⁹, G and B are as hereinbefore defined.

Within the sub-group of compounds of the formula (VII), typically G is a group R¹⁴ wherein R¹⁴ is an aryl group having six ring members and B is a bond or a methylene group, preferably a methylene group.

Preferred compounds of the formula (VII) are those wherein R⁷ is selected from hydrogen and C₁₋₄ alkyl and R⁹ is selected from hydrogen, C₁₋₄ alkyl and C₁₋₄ alkanoyl such as acetyl.

Another group of novel compounds of the invention is defined by formula (VIII): 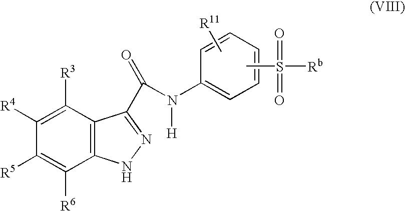
wherein R³ to R⁶ and R^{b} are as hereinbefore defined and R¹¹ represents hydrogen or one or more substituents selected from halogen, C₁₋₄ alkyl, C₁₋₄ alkoxy, trifluoromethyl and trifluoromethoxy.

In one embodiment, the group SO₂R^{b} is attached to the meta-position of the benzene ring.

In another embodiment, the group SO₂R^{b} is attached to the para-position of the benzene ring.

Preferred compounds are those in which R¹¹ is hydrogen.

In one group of compounds of the formula (VIII), R^{b} is C₁₋₄ alkyl, preferably methyl.

In another aspect, the invention provides a compound of the formula (IX): 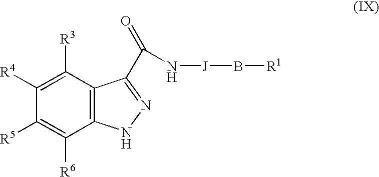
wherein

R³ to R⁶ and B are as hereinbefore defined;

J is a group R¹⁵ or CH₂—R^{15a} where R¹⁵ is a substituted or unsubstituted, non-bridged heterocyclic group having from 5 to 12 ring members, other than a diazacycloalkyl moiety, and R^{15a} is an unsubstituted or substituted aryl or heteroaryl group having from 5 to 12 ring members;

R¹ is hydrogen when R^{15a} is aryl or, when R^{15a} is other than aryl, R¹ is hydrogen or a group selected from SO₂R^{b}, SO₂NR⁷R⁸, CONR⁷R⁸, NR⁷R⁹ and carbocyclic and heterocyclic groups having from 3 to 7 ring members;

and the optional substituents for the groups R¹⁵ and R^{15a} can be one or more substituent groups R¹⁰ selected from halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

provided that when R^{15a} is aryl it is not substituted either directly, or via an acyclic linker group having a linking chain length of up to 3 atoms selected from C, N, S and O, by a group selected from SO₂Rb, SO₂NR⁷R⁸, CONR⁷R⁸, NR⁷R⁹ and carbocyclic and heterocyclic groups having from 3 to 7 ring members;

R^{c} is hydrogen or C₁₋₄ hydrocarbyl;

X¹ is O, S or NR^{c} and X² is ═O, ═S or ═NR^{c};

with the provisos that:

(a) when R¹⁵ is an azacycloalkyl group and all of R³ to R⁶ are hydrogen, at least one nitrogen atom of the azacycloalkyl group is substituted by an acyl, sulphinyl or sulphonyl group;

(b) R¹⁵ and R^{15a} are each other than a substituted or unsubstituted imidazole moiety;

but excluding the following:

(i) compounds wherein R³ and R⁶ are both hydrogen and R⁴ and R⁵ are both methoxy;

(ii) compounds wherein R³ to R⁶ are all hydrogen, J is unsubstituted pyridyl or pyridylmethyl, B is a bond and R¹ is hydrogen;

(iii) compounds wherein J is phenyl substituted with one or more of alkyl, alkoxy, alkylsulphanyl, alkylsulphinyl other than meta-alkylsulphinyl, alkylsulphonyl other than meta-alkylsulphonyl, halogen, nitro and trihalomethyl, B is a bond, and R¹ is hydrogen;

(iv) compounds wherein J is a thiophene group bearing a 3-aminocarbonyl substituent;

(v) the compound wherein J is unsubstituted phenyl or para-methoxyphenyl, and each of R³ to R⁶ is hydrogen;

(vi) N-4-methylbenzyl-1H-indazole-3-carboxamide;

(vii) compounds wherein R³, R⁵ and R⁶ are each hydrogen, R⁴ is methyl and A is unsubstituted benzyl, unsubstituted phenyl, methylphenyl, meta-trifluoromethylphenyl, and ortho-methoxyphenyl;

(viii) compounds in which J is a 2,2-dimethyl-1,3-dioxane ring;

(ix) compounds containing a benzene ring substituted by a pair of meta-oriented carboxamido moieties; and

(x) compounds wherein J is a trisubstituted phenyl group and two of the substituents are fluoro and chloro respectively.

The invention also provides a group of novel compounds of the formula (X): 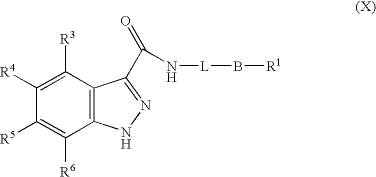
wherein

L is a group R¹⁶ or CH₂—R¹⁶ where R¹⁶ is a substituted or unsubstituted heteroaryl group other than imidazole, the heteroaryl group having from 5 to 12 ring members, at least one of which is nitrogen;

R¹ is hydrogen or a group selected from SO₂R^{b}, SO₂NR⁷R⁸, CONR⁷R⁸, NR⁷R⁹ and carbocyclic and heterocyclic groups having from 3 to 7 ring members;

B, R³, R⁴, R⁵ and R⁶ are as hereinbefore defined, provided that R⁴ and R⁵ cannot both be methoxy;

and the optional substituents for R¹⁶ can be one or more substituent groups R¹⁰ as hereinbefore defined;

but excluding compounds wherein all of R³ to R⁶ are hydrogen and L-B—R¹ defines an unsubstituted pyridyl or pyridylmethyl group.

In one general embodiment, the compound of the formulae (IX) or (X) may be other than a compound in which J is unsubstituted pyridyl or pyridylmethyl, B is a bond and R¹ is hydrogen.

Within the general formula (X), one sub-group of compounds is represented by the formula (XI): 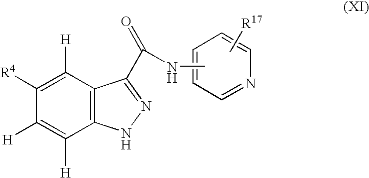
in which R¹⁷ is hydrogen, B—R¹ or R¹⁰, and wherein R⁴, B—R¹ and R¹⁰ are as hereinbefore defined, provided that at least one of R⁴ and R¹⁷ is other than hydrogen.

A preferred sub-group of compounds within formula (XI) can be represented by the formula (XII):

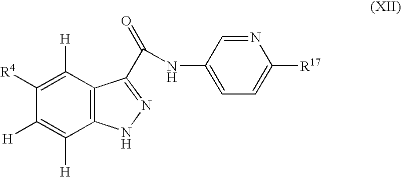

Another sub-group of compounds within the formula (X) is represented by the formula (XIII): 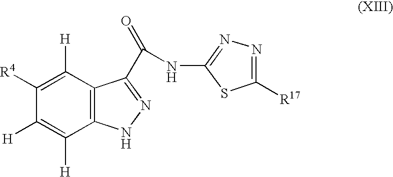
in which R¹⁷ is hydrogen, B—R¹ or R¹⁰, and wherein R⁴, B—R¹ and R¹⁰ are as hereinbefore defined.

A further sub-group of compounds within the formula (X) is represented by the formula (XIV): 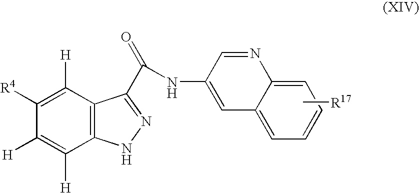
in which R¹⁷ is hydrogen, B—R¹ or R¹⁰, and wherein R⁴, B—R¹ and R¹⁰ are as hereinbefore defined.

Another group of novel compounds of the invention is the group of compounds of the formula (XV): 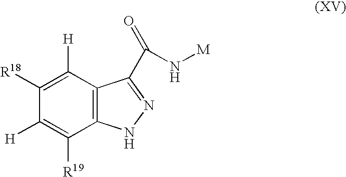
wherein

M is a group R²⁰ or CH₂—R²⁰ where R²⁰ is an aryl group having from 6 to 12 ring members and being optionally substituted by one or two substituent groups R¹⁰ which may be the same or different;

R¹⁸ is selected from hydrogen, halogen, and carbocyclic and heterocyclic groups having from 3 to 12 ring members;

R¹⁹ is selected from hydrogen and amino, provided that at least one of R¹⁸ and R¹⁹ is other than hydrogen;

provided that the aryl group R²⁰ is not substituted either directly, or via an acyclic linker group having a linking chain length of up to 3 atoms selected from C, N, S and O, by a group selected from SO₂Rb, SO₂NR⁷R⁸, CONR⁷R⁸, NR⁷R⁹ and carbocyclic and heterocyclic groups having from 3 to 7 ring members.

Preferred compounds of the formula (XV) are those wherein R¹⁸ is halogen, especially iodine or chlorine, and R¹⁹ is hydrogen.

Another group of novel compounds of the invention is the group of compounds of the formula (XVI): 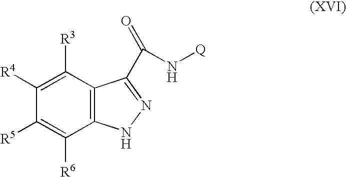
wherein

R³ to R⁶ are as hereinbefore defined;

Q is an optionally substituted non-bridged non-aromatic heterocyclic group having from 5 to 7 ring members of which at least one is a nitrogen atom, the group being other than a diazacycloalkyl group;

and the optional substituents for the group Q can be one or more (preferably up to 2, for example 1) substituent groups R²¹ selected from SO₂R^{b}, SO₂NR⁷R⁸, CONR⁷R⁸, NR⁷R⁹, halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;

R^{c} is hydrogen or C₁₋₄ hydrocarbyl;

X¹ is O, S or NR^{c} and X² is ═O, ═S or ═NR^{c};

provided that when Q is an azacycloalkyl group and R³ to R⁶ are all hydrogen, at least one nitrogen atom of the azacycloalkyl or diazacycloalkyl group is substituted by an acyl, sulphinyl or sulphonyl group.

In each of the groups of novel compounds (II) to (XVI), it is preferred that the compounds do not contain a benzene ring substituted by a pair of meta-oriented carboxamido moieties.

In the compounds of the formulae (IX) and (X), it is preferred that J-B—R¹ and L-B—R¹ are other than a diazine or triazine substituted by a monocyclic pyrazolyl group or a bicyclic fused pyrazolyl group.

In the compounds of the formulae (IX), (X) and (XVI), it is preferred that J-B—R¹ and L-B—R¹ are other than a saturated azabicyclic moiety or an imidazolyl moiety.

In compounds of the formulae (IX) and (XIV), it is preferred that when J-B—R¹ is an unsubstituted phenyl group, R³ to R⁶ are each other than a group R^{a}—R^{b} wherein R^{a} is a bond and R^{b} is a substituted C₃-C₈ hydrocarbyl group having two or more substituents, one of which contains an unsubstituted or substituted amino group.

In the foregoing definitions of novel compounds of the invention, the groups E, G, J and L are sub-groups of the group A defined in relation to compounds of the formula (I). Similarly, the groups R¹², R^{12a} and R¹⁴ are sub-groups of the group R², and the group R¹³ is a sub-group of the group R¹. Unless the context requires otherwise, the general and specific preferences, embodiments and examples set out above in relation to A, R¹ and R², apply also to the sub-groups E, G, R¹³, R¹², R^{12a} and R¹⁴.

The novel compounds of the formulae (IX) to (XVI) defined above are sub-groups of the formula (I). Except where the context dictates otherwise, the general and specific definitions of substituent groups, and the general and specific definitions, preferences and examples set out for each of the moieties R¹ to R¹⁰, A and B apply also to compounds of the formulae (IX) to (XVI).

For the avoidance of doubt, it is to be understood that each general and specific preference, embodiment and example of the groups R¹ may be combined with each general and specific preference, embodiment and example of the groups R² and/or R³ and/or R⁴ and/or R⁵ and/or R⁶ and/or R⁷ and/or R⁸ and/or R⁹ and/or R¹⁰ and/or A and/or B and their associated sub-groups, and that all such combinations are embraced by this application.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular novel compounds of the invention are as described in the Examples below.

Specific novel compounds of the invention include:
1H-Indazole-3-carboxylic acid (4-methylsulphamoylmethyl-phenyl)-amide;
1H-Indazole-3-carboxylic acid [3-(1H-tetrazol-5-yl)-phenyl]-amide;
1H-Indazole-3-carboxylic acid [4-(acetylamino-methyl)-phenyl]-amide;
1H-Indazole-3-carboxylic acid [4-(2-oxo-pyrrolidin-1-yl)-phenyl]-amide;
1H-Indazole-3-carboxylic acid (3-oxazol-5-yl-phenyl)-amide;
1H-Indazole-3-carboxylic acid [4-(1H-imidazol-4-yl)-phenyl]-amide;
1H-Indazole-3-carboxylic acid (3-methanesulphonyl-phenyl)-amide;
1H-Indazole-3-carboxylic acid [4-(morpholine-4-sulphonyl)-phenyl]-amide;
5-Iodo-1H-indazole-3-carboxylic acid (4-sulphamoyl-phenyl)-amide;
5-Iodo-1H-indazole-3-carboxylic acid (4-methylsulphamoylmethyl-phenyl)-amide;
5-Iodo-1H-indazole-3-carboxylic acid (3-methanesulphonyl-phenyl)-amide;
5-Iodo-1H-indazole-3-carboxylic acid [4-(acetylamino-methyl)-phenyl]-amide;
5-nitro-1H-indazole-3-carboxylic acid (4-sulphamoyl-phenyl)-amide;
5-nitro-1H-indazole-3-carboxylic acid (4-methylsulphamoylmethyl-phenyl)-amide;
5-thiophen-2-yl-1H-indazole-3-carboxylic acid (4-methylsulphamoylmethyl-phenyl)-amide;
5-(3,5-dimethyl-isoxazol-4-yl)-1H-indazole-3-carboxylic acid (4-methylsulphamoylmethyl-phenyl)-amide;
5-furan-2-yl-1H-indazole-3-carboxylic acid (4-methylsulphamoylmethyl-phenyl)-amide;
5-benzofuran-2-yl-1H-indazole-3-carboxylic acid (4-methylsulphamoylmethyl-phenyl)-amide;
N-phenyl-5-iodo-1H-indazole-3-carboxamide;
5-morpholin-4-yl-1H-indazole-3-carboxylic acid phenylamide;
5-chloro-1H-indazole-3-carboxylic acid (5-nitro-pyridin-2-yl)-amide;
1H-indazole-3-carboxylic acid (4-sulphamoyl-phenyl)-amide;
5-thiophen-2-yl-1H-indazole-3-carboxylic acid (4-methylsulphamoylmethyl-phenyl)-amide;
5-thiazol-2-yl-1H-indazole-3-carboxylic acid (4-methylsulphamoylmethyl-phenyl)-amide;
4-[(5-iodo-1H-indazole-3-carbonyl)-amino]-piperidine-1-carboxylic acid ethyl ester;
1H-indazole-3-carboxylic acid [4-(thiazol-2-ylsulphamoyl)-phenyl]-amide;
5-phenyl-1H-indazole-3-carboxylic acid (4-methylsulphamoylmethyl-phenyl)-amide;
5-nitro-1H-indazole-3-carboxylic acid [4-(methanesulphonylamino-methyl)-phenyl]-amide;
4-[(5-nitro-1H-indazole-3-carbonyl)-amino]-piperidine-1-carboxylic acid ethyl ester;
5-chloro-1H-indazole-3-carboxylic acid (1-benzyl-pyrrolidin-3-yl)-amide;
4-[(5-chloro-1H-indazole-3-carbonyl)-amino]-piperidine-1-carboxylic acid ethyl ester;
5-iodo-1H-indazole-3-carboxylic acid (6-methoxy-pyridin-3-yl)-amide;
5-iodo-1H-indazole-3-carboxylic acid pyridin-3-yl-amide;
5-iodo-1H-indazole-3-carboxylic acid quinolin-3-ylamide;
5-iodo-1H-indazole-3-carboxylic acid (tetrahydro-pyran-4-yl)-amide;
5-chloro-1H-indazole-3-carboxylic acid (1-methyl-piperidin-4-yl)-amide;
5-iodo-1H-indazole-3-carboxylic acid (2-chloro-pyridin-3-yl)-amide;
5-chloro-1H-indazole-3-carboxylic acid benzylamide;
5-chloro-1H-indazole-3-carboxylic acid 4-(4-methyl-piperazin-1-yl)-benzylamide;
5-chloro-1H-indazole-3-carboxylic acid pyridin-3-ylamide;
5-iodo-1H-indazole-3-carboxylic acid (6-cyano-pyridin-3-yl)-amide;
5-chloro-1H-indazole-3-carboxylic acid phenylamide;
5-iodo-1H-indazole-3-carboxylic acid (6-methyl-pyridazin-3-yl)-amide;
5-chloro-1H-indazole-3-carboxylic acid (5-ethyl-[1,3,4]thiadiazol-2-yl)-amide;
5-iodo-1H-indazole-3-carboxylic acid (4-morpholin-4-yl-phenyl)-amide;
5-iodo-1H-indazole-3-carboxylic acid (2-oxo-1,2-dihydro-pyridin-3-yl)-amide;
1H-indazole-3-carboxylic acid (4-morpholin-4-yl-phenyl)-amide;
5-nitro-1H-indazole-3-carboxylic acid phenylamide;
5-iodo-1H-indazole-3-carboxylic acid (6-chloro-pyridin-3-yl)-amide;
4-[(1H-indazole-3-carbonyl)-amino]-piperidine-1-carboxylic acid tert-butyl ester;
5-iodo-1H-indazole-3-carboxylic acid (4-fluoro-phenyl)-amide;
5-iodo-1H-indazole-3-carboxylic acid (6-acetylamino-pyridin-3-yl)-amide;
5-amino-1H-indazole-3-carboxylic acid phenylamide;
5-iodo-1H-indazole-3-carboxylic acid (4-methylaminosulphonylmethyl-phenyl)-amide;
5-amino-1H-indazole-3-carboxylic acid (4-sulphamoyl-phenyl)-amide;
7-amino-1H-indazole-3-carboxylic acid (4-sulphamoyl-phenyl)-amide;
5-[3-(2-chloro-ethyl)-ureido]-1H-indazole-3-carboxylic acid (4-methylsulphamoyl-methyl-phenyl)-amide;
5-nitro-1H-indazole-3-carboxylic acid (4-methylsulphamoylmethyl-phenyl)-amide;
5-amino-1H-indazole-3-carboxylic acid (4-methylsulphamoylmethyl-phenyl)-amide;
5-iodo-1H-indazole-3-carboxylic acid piperidin-4-ylamide
5-chloro-1H-indazole-3-carboxylic acid [4-(acetylamino-methyl)-phenyl]-amide;
1H-indazole-3-carboxylic acid [1-(2,2,2 trifluoro-acetyl)-Piperidin-4-yl]-amide;
1H-indazole-3-carboxylic acid piperidin-4-ylamide;
1H-indazole-3-carboxylic acid (1-acetyl-piperidin-4-yl)-amide;
1H-indazole-3-carboxylic acid (1-methanesulphonyl-piperidin-4-yl)-amide;
1H-indazole-3-carboxylic acid (4-fluoro-phenyl)-amide;
4-bromo-1H-indazole-3-carboxylic acid (4-fluoro-phenyl)-amide;
5-nitro-1H-indazole-3-carboxylic acid (4-fluorophenyl)-amide;
5-amino-1H-indazole-3-carboxylic acid (4-fluorophenyl)-amide;
5-amino-4-bromo-1H-indazole-3-carboxylic acid (4-fluorophenyl)-amide;
5-methyl-1H-indazole-3-carboxylic acid (4-fluoro-phenyl)-amide;
6-bromo-1H-indazole-3-carboxylic acid (4-fluoro-phenyl)-amide;
5-chloro-1H-indazole-3-carboxylic acid (4-morpholin-4-yl-phenyl)-amide;
5-chloro-1H-indazole-3-carboxylic acid [3-(1H-tetrazol-5-yl)-phenyl]-amide;
5-iodo-1H-indazole-3-carboxylic acid (4-pyrrolidin-1-ylmethyl-phenyl)-amide;
5-chloro-1H-indazole-3-carboxylic acid [4-(thiazol-2-ylsulphamoyl)-phenyl]-amide;
5-chloro-1H-indazole-3-carboxylic acid (4-fluoro-phenyl)-amide;
3-[(5-chloro-1H-indazole-3-carbonyl)-amino]-pyrrolidine-1-carboxylic acid methyl ester;
5-fluoro-1H-indazole-3-carboxylic acid phenylamide;
5-morpholin-4-yl-1H-indazole-3-carboxylic acid (6-chloro-pyridin-3-yl)-amide;
1H-indazole-3-carboxylic acid (6-chloro-pyridin-3-yl)-amide;
5-phenethyl-1H-indazole-3-carboxylic acid phenylamide;
5-(1,1-dioxo-1 lambda*6*-isothiazolidin-2-yl)-1H-indazole-3-carboxylic acid phenylamide;
5-biphenyl-2-yl-1H-indazole-3-carboxylic acid phenylamide;
5-pyrrolidin-1-yl-1H-indazole-3-carboxylic acid phenylamide;
5-chloro-1H-indazole-3-carboxylic acid [5-(tetrahydro-furan-2-yl)-[1,3,4]thiadiazol-2-yl]-amide and
5-nitro-1H-indazole-3-carboxylic acid (3-methanesulphonyl-phenyl)-amide.

Pharmaceutical compositions comprising a novel compound as hereinbefore defined and a pharmaceutically acceptable carrier also form part of the invention.

The invention also provides a novel compound as hereinbefore defined for use in medicine, for example for one or more of the uses set out above in relation to compounds of the formula (I).

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds.

Acid addition salts may be formed with a wide variety of acids, both inorganic and organic. Examples of acid addition salts include salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

If the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH⁴⁺) and substituted ammonium ions (e.g., NH₃R+, NH₂R₂₊, NHR³⁺, NR⁴⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

Esters such as carboxylic acid esters and acyloxy esters of the compounds of formula (I) bearing a carboxylic acid group or a hydroxyl group are also embraced by Formula (I). Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group.

Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl

(e.g., -Me, -Et, -nPr, -iPr, -nBu, -sBu, -iBu, -tBu);

C₁₋₇aminoalkyl

(e.g., aminoethyl; 2-(N,N-diethylamino)ethyl; 2-(4-morpholino)ethyl); and

acyloxy-C₁₋₇alkyl

(e.g., acyloxymethyl;

acyloxyethyl;

pivaloyloxymethyl; acetoxymethyl;

1-acetoxyethyl;

1-(1-methoxy-1-methyl)ethyl-carbonxyloxyethyl;

1-(benzoyloxy)ethyl; isopropoxy-carbonyloxymethyl;

1-isopropoxy-carbonyloxyethyl; cyclohexyl-carbonyloxymethyl;

1-cyclohexyl-carbonyloxyethyl;

cyclohexyloxy-carbonyloxymethyl;

1-cyclohexyloxy-carbonyloxyethyl;

(4-tetrahydropyranyloxy) carbonyloxymethyl;

1-(4-tetrahydropyranyloxy)carbonyloxyethyl;

(4-tetrahydropyranyl)carbonyloxymethyl; and

1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Where the compounds of the formula (I) contain chiral centres, all individual optical forms such as enantiomers, epimers and diastereoisomers, as well as racemic mixtures of the compounds are within the scope of formula (I).

The compounds of the formula (I) are inhibitors of cyclin dependent kinases. As such, they are expected to be useful in providing a means of arresting, or recovering control of, the cell cycle in abnormally dividing cells. It is therefore anticipated that the compounds will prove useful in treating or preventing proliferative disorders such as cancers. It is also envisaged that the compounds of the invention will be useful in treating conditions such as viral infections, autoimmune diseases and neurodegenerative diseases for example.

CDKs play a role in the regulation of the cell cycle, apoptosis, transcription, differentiation and CNS function. Therefore, CDK inhibitors could be useful in the treatment of diseases in which there is a disorder of proliferation, apoptosis or differentiation such as cancer. In particular RB+ve tumours may be particularly sensitive to CDK inhibitors.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma; a hematopoietic tumor of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumor of the central or peripheral nervous system, for example astrocytoma, neuroblastoma, glioma or schwannoma; melanoma; seminoma; teratocarcinoma; osteosarcoma; xenoderoma pigmentoum; keratoctanthoma; thyroid follicular cancer; or Kaposi's sarcoma.

CDKs are also known to play a role in apoptosis, proliferation, differentiation and transcription and therefore CDK inhibitors could also be useful in the treatment of the following diseases other than cancer; viral infections, for example herpes virus, pox virus, Epstein-Barr virus, Sindbis virus, adenovirus, HIV, HPV, HCV and HCMV; prevention of AIDS development in HIV-infected individuals; chronic inflammatory diseases, for example systemic lupus erythematosus, autoimmune mediated glomerulonephritis, rheumatoid arthritis, psoriasis, inflammatory bowel disease, and autoimmune diabetes mellitus; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, arrhythmia, atherosclerosis, toxin-induced or alcohol related liver diseases, haematological diseases, for example, chronic anemia and aplastic anemia; degenerative diseases of the musculoskeletal system, for example, osteoporosis and arthritis, aspirin-senstive rhinosinusitis, cystic fibrosis, multiple sclerosis, kidney diseases and cancer pain.

It has also been discovered that some cyclin-dependent kinase inhibitors can be used in combination with other anticancer agents. For example, the cytotoxic activity of cyclin-dependent kinase inhibitor flavopiridol, has been used with other anticancer agents in combination therapy.

Thus, in the pharmaceutical compositions, uses or methods of this invention for treating a disease or condition comprising abnormal cell growth, the disease or condition comprising abnormal cell growth in one embodiment is a cancer.

Particular subsets of cancers include breast cancer, ovarian cancer, colon cancer, prostate cancer, oesophageal cancer, squamous cancer and non-small cell lung carcinomas.

Methods for the Preparation of Compounds of the Formula (1)

Compounds of the formula (I) and the various sub-groups thereof as hereinbefore defined can be prepared by reacting an amine of the formula H₂N-A-B—R¹ with an indazole 3-carboxylic acid of the formula (XVII):

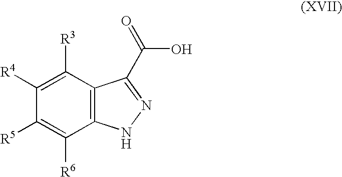

wherein R³ to R⁶ are as hereinbefore defined. The coupling reaction between the amine and the carboxylic acid (XVII) can be carried out by forming an activated derivative of the acid such as an acid chloride (e.g. by reaction with thionyl chloride), and then reacting the acid chloride with the amine, for example by the method described in Zh. Obs. Khim. 31, 201 (1961), and the method described in U.S. Pat. No. 3,705,175.

Alternatively, and more preferably, the coupling reaction between the carboxylic acid (XVII) and the amine can be carried out in the presence of an amide coupling reagent of the type commonly used to form peptide linkages. Examples of such reagents include 1,3-dicyclohexylcarbodiimide (DCC) (Sheehan et al, J. Amer. Chem Soc. 1955, 77, 1067), 1-ethyl-3-(3′-dimethylaminopropyl)carbodiimide (EDCI) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) (L. A. Carpino, J. Amer. Chem. Soc., 1993, 115, 4397) and phosphonium-based coupling agents such as 1-benzo-triazolyloxytris(pyrrolidino)phosphonium hexafluorophosphate (PyBOP) (Castro et al, Tetrahedron Letters, 1990, 31, 205). A preferred coupling reagent is HATU. Carbodiimide-based coupling agents are advantageously used in combination with 1-hydroxybenzotriazole (HOBt) (Konig et al, Chem. Ber., 103, 708, 2024-2034). Preferred coupling reagents include EDC and DCC in combination with HOBt.

The coupling reaction is typically carried out in a non-aqueous, non-protic solvent such as dichloromethane, dimethylformamide or N-methylpyrrolidine. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

Carboxylic acids of the formula (XVII) can be obtained commercially. Alternatively, compounds of the formula (XVII) can be prepared from compounds of the formula (XVIII): 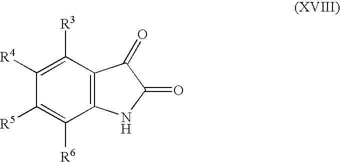
by a sequence of reactions involving ring-opening, diazotisation, reduction and cyclisation. Ring opening of the substituted isatin compound to give an ortho-aminophenyl-glyoxylic acid derivative can be achieved using an aqueous alkali such as sodium hydroxide with moderate heating, for example to a temperature of 35° C. The amine can then be converted to the diazonium salt by treatment with nitrous acid (for example generated from sodium nitrite and sulphuric acid) at a reduced temperature (e.g. approximately 5° C.). The diazonium salt is reduced to form a hydrazine using a reducing agent such as tin (II) chloride and is then cyclised to the indazole by a cyclo-condensation reaction.

Isatin derivatives of the formula (XVIII) are available commercially or can be prepared by a variety of known methods.

For example, according to the method described by Hewawasam et al, Tetrahedron Letters, 1994, 35, 7303-7306, N-protected anilines can be subjected to ortho-lithiation and the lithiated intermediate reacted with diethyl oxalate to give an α-ketoester which cyclises to give an isatin upon deprotection of the amino group.

According to the method of Garden et al, Tetrahedron Letters, 1997, 38, 1501-1504, substituted anilines an be reacted with trichloroacetaldehyde and hydroxylamine in the presence of acid to give an α-isonitrosoacetanilide which cyclises to give an isatin.

According to the method of Kraynack et al, Tetrahedron Letters, 1998, 39, 7679-7682, substituted isatins can be formed by the γ-dibromination of 2-oxo-indolines and subsequent hydrolysis of the resulting dibromo-compounds.

An alternative route to compounds of the formula (I) involves the reaction of a substituted phenyl acetic acid amide compound of the formula (XIX): 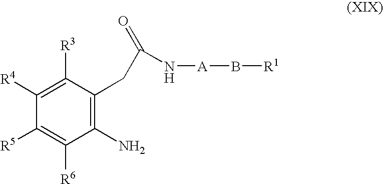
with nitrous acid or an alkyl nitrite at a reduced temperature (e.g. lower than 20° C. and preferably below 0° C.) in the presence of a mineral acid such as hydrochloric acid or sulphuric acid or a mixture of hydrochloric acid and acetic acid, for example as described in U.S. Pat. No. 3,705,175.

Compounds of the formula (XIX) can be prepared inter alia by reduction of the corresponding ortho-nitrophenylacetyl compound, for example under conditions analogous to those described in Morie et al, Synth. Commun., 1997, 27, 559-566.

Compounds of the formula (I) can also be prepared from other compounds of the formula (I) bearing suitable substituents and suitable reactive groups. For example, compounds wherein one or more of R³ to R⁶ are bromine or iodine, particularly iodine, can be used as intermediates for the preparation of other compounds of the formula (I).

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2(-phenylsulphonyl)ethyloxy amide (—NH-Psec). Other protecting groups for amines, such as cyclic amines and heterocyclic N—H groups, include toluenesulphonyl (tosyl) and methanesulphonyl (mesyl) groups and benzyl groups such as a para-methoxybenzyl (PMB) group. A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

A more detailed description of the processes that can be used to prepare the compounds of the formula (I) can be found in the specific examples set out below.

Pharmaceutical Formulations

The invention also provides compounds of the formula (I) as hereinbefore defined in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastrointestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the inventions will generally be presented in unit dosage form and, as such, will typically contain sufficient compound to provide a desired level of biological activity. For example, a formulation intended for oral administration may contain from 0.1 milligrams to 2 grams of active ingredient, more usually from 10 milligrams to 1 gram, for example, 50 milligrams to 500 milligrams.

The active compound will be administered to a patient in need thereof (for example a human or animal patient) in an amount sufficient to achieve the desired therapeutic effect.

Methods of Treatment

It is envisaged that the compounds of the formula (I) will useful in the prophylaxis or treatment of a range of disease states or conditions mediated by cyclin dependent kinases. Examples of such disease states and conditions are set out above.

Compounds of the formula (I) are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

A typical daily dose of the compound can be in the range from 100 picograms to 100 milligrams per kilogram of body weight, more typically 10 nanograms to 10 milligrams per kilogram of bodyweight although higher or lower doses may be administered where required. Ultimately, the quantity of compound administered will be commensurate with the nature of the disease or physiological condition being treated and will be at the discretion of the physician.

The compounds of the formula (I) can be administered as the sole therapeutic agent or they can be administered in combination therapy with one of more other compounds for treatment of a particular disease state, for example a neoplastic disease such as a cancer as hereinbefore defined. Examples of other therapeutic agents that may be administered together (whether concurrently or at different time intervals) with the compounds of the formula (I) include cytotoxic agents, agents that prevent cell proliferation or radiotherapy. Examples of such agents include but are not limited to topoisomerase inhibitors, alkylating agents, antimetabolites, DNA binders and microtubule inhibitors, such as cisplatin, cyclophosphamide, doxorubicin, irinotecan, fludarabine, 5FU, taxanes and mitomycin C.

Antifungal Use

In a further aspect, the invention provides the use of the compounds of the formula (I) as hereinbefore defined as antifungal agents.

The compounds of the formula (I) may be used in animal medicine (for example in the treatment of mammals such as humans), or in the treatment of plants (e.g. in agriculture and horticulture), or as general antifungal agents, for example as preservatives and disinfectants.

In one embodiment, the invention provides a compound of the formula (I) as hereinbefore defined for use in the prophylaxis or treatment of a fungal infection in a mammal such as a human.

Also provided is the use of a compound of the formula (I) for the manufacture of a medicament for use in the prophylaxis or treatment of a fungal infection in a mammal such as a human.

For example, compounds of the invention may be administered to human patients suffering from, or at risk of infection by, topical fungal infections caused by among other organisms, species of Candida, Trichophyton, Microsporum or Epidermophyton, or in mucosal infections caused by Candida albicans (e.g. thrush and vaginal candidiasis). The compounds of the invention can also be administered for the treatment or prophylaxis of systemic fungal infections caused by, for example, Candida albicans, Cryptococcus neoformans, Aspergillus flavus, Aspergillus fumigatus, Coccidiodies, Paracoccidioides, Histoplasma or Blastomyces.

In another aspect, the invention provides an antifungal composition for agricultural (including horticultural) use, comprising a compound of the formula (I) together with an agriculturally acceptable diluent or carrier.

The invention further provides a method of treating an animal (including a mammal such as a human), plant or seed having a fungal infection, which comprises treating said animal, plant or seed, or the locus of said plant or seed, with an effective amount of a compound of the formula (I).

The invention also provides a method of treating a fungal infection in a plant or seed which comprises treating the plant or seed with an antifungally effective amount of a fungicidal composition as hereinbefore defined.

Differential screening assays may be used to select for those compounds of the present invention with specificity for non-human CDK enzymes. Compounds which act specifically on the CDK enzymes of eukaryotic pathogens can be used as anti-fungal or anti-parasitic agents. Inhibitors of the Candida CDK kinase, CKSI, can be used in the treatment of candidiasis. Antifungal agents can be used against infections of the type hereinbefore defined, or opportunistic infections that commonly occur in debilitated and immunosuppressed patients such as patients with leukemias and lymphomas, people who are receiving immunosuppressive therapy, and patients with predisposing conditions such as diabetes mellitus or AIDS, as well as for non-immunosuppressed patients.

Assays described in the art can be used to screen for agents which may be useful for inhibiting at least one fungus implicated in mycosis such as candidiasis, aspergillosis, mucormycosis, blastomycosis, geotrichosis, cryptococcosis, chromoblastomycosis, coccidiodomycosis, conidiosporosis, histoplasmosis, maduromycosis, rhinosporidosis, nocaidiosis, para-actinomycosis, penicilliosis, monoliasis, or sporotrichosis. The differential screening assays can be used to identify anti-fungal agents which may have therapeutic value in the treatment of aspergillosis by making use of the CDK genes cloned from yeast such as Aspergillus fumigatus, Aspergillus flavus, Aspergillus niger, Aspergillus nidulans, or Aspergillus terreus, or where the mycotic infection is mucon-nycosis, the CDK assay can be derived from yeast such as Rhizopus arrhizus, Rhizopus oryzae, Absidia corymbifera, Absidia ramosa, or Mucorpusillus. Sources of other CDK enzymes include the pathogen Pneumocystis carinii.

By way of example, in vitro evaluation of the antifungal activity of the compounds can be performed by determining the minimum inhibitory concentration (M.I.C.) which is the concentration of the test compounds, in a suitable medium, at which growth of the particular microorganism fails to occur. In practice, a series of agar plates, each having the test compound incorporated at a particular concentration is inoculated with a standard culture of, for example, Candida albicans and each plate is then incubated for an appropriate period at 37° C. The plates are then examined for the presence or absence of growth of the fungus and the appropriate M.I.C. value is noted

The in vivo evaluation of the compounds can be carried out at a series of dose levels by intraperitoneal or intravenous injection or by oral administration, to mice that have been inoculated with a fungus, e.g., a strain of Candida albicans or Aspergillus flavus. The activity of the compounds can be assessed on the basis of the survival of a treated group of mice after the death of an untreated group of mice. The activity may be measured in terms of the dose level at which the compound provides 50% protection against the lethal effect of the infection (PD₅₀).

For human antifungal use, the compounds of the formula (I) can be administered alone or in admixture with a pharmaceutical carrier selected in accordance with the intended route of administration and standard pharmaceutical practice. Thus, for example, they may be administered orally, parenterally, intravenously, intramuscularly or subcutaneously by means of the formulations described above in the section headed “Pharmaceutical Formulations”.

For oral and parenteral administration to human patients, the daily dosage level of the antifungal compounds of the formula (I) be from 0.01 to 10 mg/kg (in divided doses), depending on inter alia the potency of the compounds when administered by either the oral or parenteral route. Tablets or capsules of the compounds may contain, for example, from 5 mg. to 0.5 g of active compound for administration singly or two or more at a time as appropriate. The physician in any event will determine the actual dosage (effective amount) which will be most suitable for an individual patient and it will vary with the age, weight and response of the particular patient.

Alternatively, the antifungal compounds of formula (I) can be administered in the form of a suppository or pessary, or they may be applied topically in the form of a lotion, solution, cream, ointment or dusting powder. For example, they can be incorporated into a cream consisting of an aqueous emulsion of polyethylene glycols or liquid paraffin; or they can be incorporated, at a concentration between 1 and 10%, into an ointment consisting of a white wax or white soft paraffin base together with such stabilizers and preservatives as may be required.

In addition to the therapeutic uses described above, anti-fungal agents developed with such differential screening assays can be used, for example, as preservatives in foodstuff, feed supplement for promoting weight gain in livestock, or in disinfectant formulations for treatment of non-living matter, e.g., for decontaminating hospital equipment and rooms. In similar fashion, side by side comparison of inhibition of a mammalian CDK and an insect CDK, such as the Drosophilia CDK5 gene (Hellmich et al. (1994) FEBS Lett 356:317-21), will permit selection amongst the compounds herein of inhibitors which discriminate between the human/mammalian and insect enzymes. Accordingly, the present invention expressly contemplates the use and formulations of the compounds of the invention in insecticides, such as for use in management of insects like the fruit fly.

In yet another embodiment, certain of the subject CDK inhibitors can be selected on the basis of inhibitory specificity for plant CDK's relative to the mammalian enzyme. For example, a plant CDK can be disposed in a differential screen with one or more of the human enzymes to select those compounds of greatest selectivity for inhibiting the plant enzyme. Thus, the present invention specifically contemplates formulations of the subject CDK inhibitors for agricultural applications, such as in the form of a defoliant or the like.

For agricultural and horticultural purposes the compounds of the invention may be used in the form of a composition formulated as appropriate to the particular use and intended purpose. Thus the compounds may be applied in the form of dusting powders, or granules, seed dressings, aqueous solutions, dispersions or emulsions, dips, sprays, aerosols or smokes. Compositions may also be supplied in the form of dispersible powders, granules or grains, or concentrates for dilution prior to use. Such compositions may contain such conventional carriers, diluents or adjuvants as are known and acceptable in agriculture and horticulture and they are manufactured in accordance with conventional procedures. The compositions may also incorporate other active ingredients, for example, compounds having herbicidal or insecticidal activity or a further fungicide. The compounds and compositions can be applied in a number of ways, for example they can be applied directly to the plant foliage, stems, branches, seeds or roots or to the soil or other growing medium, and they may be used not only to eradicate disease, but also prophylactically to protect the plants or seeds from attack. By way of example, the compositions may contain from 0.01 to 1 wt. % of the active ingredient. For field use, likely application rates of the active ingredient may be from 50 to 5000 g/hectare.

The invention also contemplates the use of the compounds of the formula (I) in the control of wood decaying fungi and in the treatment of soil where plants grow, paddy fields for seedlings, or water for perfusion. Also contemplated by the invention is the use of the compounds of the formula (I) to protect stored grain and other non-plant loci from fungal infestation.

### EXAMPLES

The invention will now be illustrated, but not limited, by reference to the specific embodiments described in the following examples.

In the examples, the compounds prepared were characterised by liquid chromatography and mass spectroscopy using two systems, the details of which are set out below. Where chlorine is present, the mass quoted for the compound is for ³⁵Cl unless otherwise indicated. The two systems were equipped with identical chromatography columns and were set up to run under the same operating conditions. The operating conditions used are also described below.

1. Platform System

System: Waters 2790/Platform LC

Mass Spec Detector: Micromass Platform LC

PDA Detector: Waters 996 PDA

Analytical Conditions:

Eluent A: H₂O (1% Formic Acid)

Eluent B: CH₃CN (1% Formic Acid)

Gradient: 5-95% eluent B

Flow: 1.5 ml/min

Column: Synergi 4 μm Max-RP C₁₂, 80A, 50×4.6 mm (Phenomenex)

MS Conditions:

Capillary voltage: 3.5 kV

Cone voltage: 30 V

Source Temperature: 120° C.

2. FractionLynx System

System: Waters FractionLynx (dual analytical/prep)

Mass Spec Detector: Waters-Micromass ZQ

PDA Detector: Waters 2996 PDA

Analytical Conditions:

Eluent A: H₂O (1% Formic Acid)

Eluent B: CH₃CN (1% Formic Acid)

Gradient: 5-95% eluent B

Flow: 1.5 ml/min

Column: Synergi 4 μm Max-RP C₁₂, 80A, 50×4.6 mm (Phenomenex)

MS Conditions:

Capillary voltage: 3.5 kV

Cone voltage: 30 V

Source Temperature: 120° C.

Desolvation Temperature: 230° C.

The starting materials for each of the Examples are commercially available unless otherwise specified.

### Example 1

General Amide Preparative Procedure A

To a solution of indazole-3-carboxylic acid (Fluka) (405 mg, 2.5 mmol, 1.0 equiv) in dichloromethane (10 ml) was added an amine or appropriately substituted aniline (3.0 mmol, 1.2 equiv), N,N-diisopropylethylamine (1.6 ml, 9.0 mmol, 3.6 equiv) and O-(7-azabenzotriazol-1-yl)-N,N,N′,N′-tetramethyluronium hexafluorophosphate (1.05 g, 2.75 mmol, 1.1 equiv). The mixture was stirred for a period of 24-72 hours and additional O-(7-azabenzotriazol-1-yl)-N,N,N′,N′-tetramethyluronium hexafluorophosphate was added if necessary. The reaction was quenched with water (10 ml) and dichloromethane (10 ml). The compounds were purified as described in the examples below, and characterised by liquid chromatography and mass spectrometry using either of the systems described above.

### Example 2

General Amide Preparative Procedure B

To a suspension of 5-iodoisatin (Lancaster Synthesis) (2.2 g, 8.0 mmol, 1.0 equiv) or 5-chloroisatin (Lancaster Synthesis) (1.0 equiv.) in water (20 ml) was added NaOH (0.34 g, 8.48 mmol, 1.06 equiv) and the mixture was warmed to approximately 35° C. for 30 minutes to form a solution. The solution was cooled to 5° C. and a solution of sodium nitrite (0.62 g, 8.98 mmol, 1.12 equiv) was added dropwise over approximately 30 minutes, keeping the temperature below 10° C. The whole mixture was added dropwise via a cannula to a vigorously stirred solution of concentrated sulphuric acid (1.53 g, 15.6 mmol, 1.95 equiv) in water (20 ml) keeping the temperature below 10° C. The mixture was stirred for 20 minutes and a solution of tin (II) chloride (3.7 g, 19.52 mmol, 2.44 equiv) in concentrated hydrochloric acid (8 ml) was added dropwise. The mixture was stirred at 5° C. for 2 hours and the resulting crude 5-iodo or 5-chloro indazole-3-carboxylic acid (a yellow solid) was isolated by filtration and washed several times with water. The yellow solid was then azeotroped with toluene (3×100 ml) to remove water prior to the next step. The crude product was dissolved in dichloromethane (36 ml) and split into four 8 ml portions. To the separate solutions of crude 5-iodo or 5-chloro indazole-3-carboxylic acid in dichloromethane (8 ml) was added the appropriate amine/aniline (2.4 mmol, 1.2 equiv), N,N-diisopropylethylamine (1.2 ml, 7.2 mmol, 3.6 equiv) and O-(7-azabenzotriazol-1-yl)-N,N,N′,N′-tetramethyluronium hexafluorophosphate (0.84 g, 2.20 mmol, 1.1 equiv). The mixture was stirred for a period of 24-72 hours and was then quenched with water (8 ml) and dichloromethane (8 ml). The compounds were purified as described in the examples below, and characterised by liquid chromatography and mass spectrometry using either of the systems described above.

By following either preparative Procedure A or Procedure B, compounds of the formula (I) were prepared as described in Examples 3 to 14.

### Example 3

### N-[4-(Methylsulphonylaminomethyl)phenyl]-1H-indazole-3-carboxamide

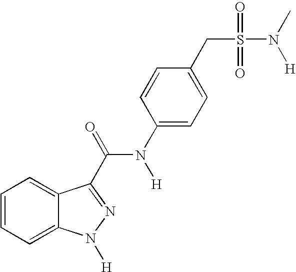

Procedure A was followed. Water and dichloromethane were removed by filtration and the solid was triturated with water and dichloromethane. The title compound was dried in vacuo to afford 119 mg (14%); LCMS 2.92 min, m/z [M+H]⁺ 345.

### Example 4

### Preparation of N-[3-(1H-tetrazol-5-yl)phenyl]-1H-indazole-3-carboxamide

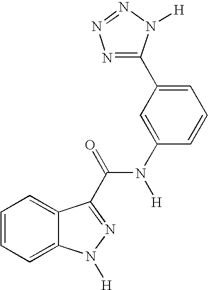

Procedure A was followed. The water and dichloromethane layers were separated and the aqueous layer was acidified with 2N HCl to form a precipitate. The precipitate was filtered. The title compound was dried in vacuo to afford 119 mg (14%); LCMS 2.95 min, m/z [M+H]⁺ 306.

### Example 5

### Preparation of N-[4-(acetylaminomethyl)phenyl]-1H-indazole-3-carboxamide

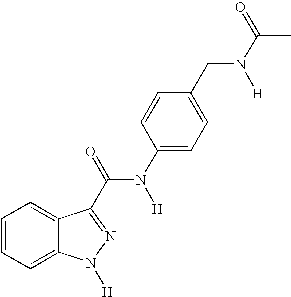

Procedure A was followed. Water and dichloromethane were removed by filtration and the solid was triturated with water and dichloromethane. The title compound was dried in vacuo to afford 190 mg (25%); LCMS 2.68 min, m/z [M+H]⁺ 309.

### Example 6

### Preparation of acid N-[4-(2-oxopyrrolidin-1-yl)phenyl]-1H-indazole-3-carboxamide

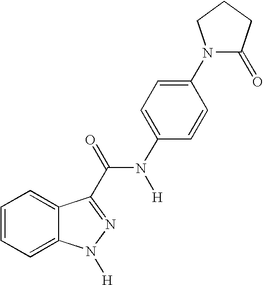

Procedure A was followed. Water and dichloromethane were removed by filtration and the solid was triturated with water and dichloromethane. The title compound was dried in vacuo to afford 311 mg (39%); LCMS 3.00 min, m/z [M+H]⁺ 321.

### Example 7

### Preparation of N-[3-(oxazol-5-yl)phenyl)-1H-indazole-3-carboxamide

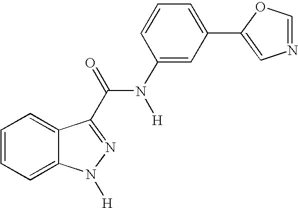

Procedure A was followed. Water and dichloromethane were removed by filtration and the solid was triturated with water and dichloromethane. The title compound was dried in vacuo to afford 276 mg (36%); LCMS 3.42 min, m/z [M+H]⁺ 305.

### Example 8

### Preparation of N-[4-(1H-imidazol-4-yl)phenyl]-1H-indazole-3-carboxamide

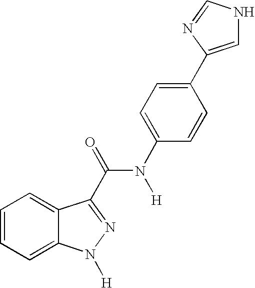

Procedure A was followed. Water and dichloromethane were removed by filtration and the solid was triturated with water and dichloromethane. The title compound was further purified by preparative HPLC to afford 1 mg (1%); LCMS 1.99 min, m/z [M+H]+304.

### Example 9

### Preparation of N-[3-methanesulphonylphenyl]-1H-indazole-3-carboxamide

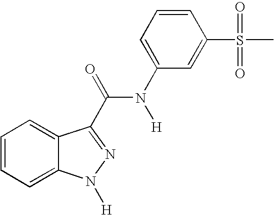

Procedure A was followed. The layers were separated and the aqueous layer was extracted twice with dichloromethane. The combined organic layers were washed with brine, dried (MgSO₄) and concentrated under reduced pressure. The title compound was purified by chromatography (SiO₂), eluting with 50% ethyl acetate-petrol, to afford 114 mg (14%); LCMS 3.09 min, m/z [M+H]⁺ 316.

### Example 10

### Preparation of N-[4-(morpholine-4-sulphonyl)phenyl]-1H-indazole-3-carboxamide

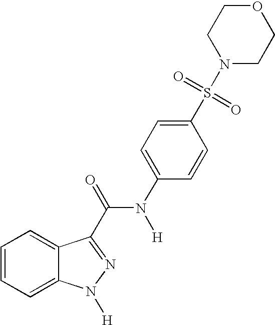

Procedure A was followed. Water and dichloromethane were removed by filtration and the solid was triturated with water and dichloromethane. The title compound was further purified by preparative HPLC to afford 18 mg (2%); LCMS 3.39 min, m/z [M+H]⁺ 387.

### Example 11

### Preparation of N-phenyl-5-iodo-1H-indazole-3-carboxamide

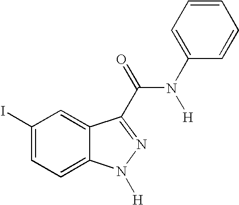

Procedure B was followed. Water and dichloromethane were removed by filtration and the solid was triturated with water and dichloromethane. The title compound was dried in vacuo to afford 53 mg (7%); LCMS 4.11 min, m/z [M+H]⁺ 364.

### Example 12

### Preparation of N-(4-aminosulphonylphenyl)-5-iodo-1H-indazole-3-carboxamide

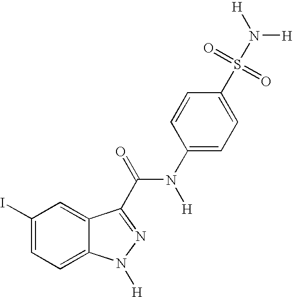

Procedure B was followed. Water and dichloromethane were removed by filtration and the solid was triturated with water and dichloromethane. The title compound was dried in vacuo to afford 16 mg (2%); LCMS 3.30 min, m/z [M+H]⁺ 443.

### Example 13

### Preparation of N-[4-(methylaminosulphonylmethyl)phenyl)]-5-iodo-1H-indazole-3-carboxamide

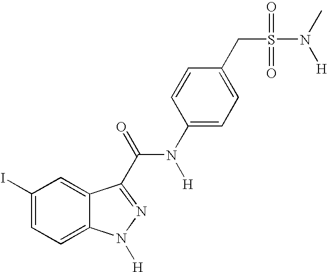

Procedure B was followed. Water and dichloromethane were removed by filtration and the solid was triturated with water and dichloromethane. The title compound was dried in vacuo to afford 21 mg (2%); LCMS 3.48 min, m/z [M+H]⁺ 471.

### Example 14

### Preparation of N-(3-methanesulphonylphenyl)-5-iodo-1H-indazole-3-carboxamide

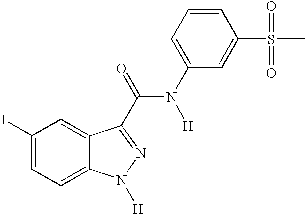

Procedure B was followed. Water and dichloromethane were removed by filtration and the solid was triturated with water and dichloromethane. The title compound was further purified by preparative HPLC to afford 2 mg (1%); LCMS 4.02 min, m/z [M+H]⁺ 442.

### Example 15

### Preparation of N-[4-(acetylaminomethyl)phenyl]-5-iodo-1H-indazole-3-carboxamide

### 15A. Preparation of N-(4-amino-benzyl)-acetamide

To 4-aminobenzylamine (3.4 ml, 30.0 mmol, 1.0 equiv) was added pyridine (30 ml) and acetic anhydride (3.1 ml, 33.0 mmol, 1.1 equiv). The mixture was stirred at room temperature for 3 days. The reaction mixture was quenched with water and the aqueous phase was extracted with EtOAc (2×). The combined organic layers were washed with brine, dried (MgSO₄) and concentrated under reduced pressure. The title compound was purified by Biotage (SiO₂, 100 g) eluting with 100% EtOAc to afford 1.47 g (30%) of the title compound.

### 15B. N-[4-(acetylaminomethyl)phenyl]-5-iodo-1H-indazole-3-carboxamide

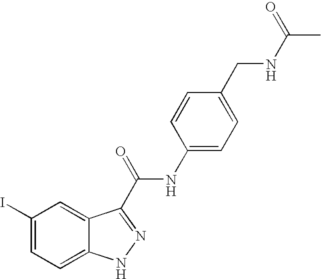

Procedure B was followed using the amine produced in 15A. Water and dichloromethane were removed by filtration and the solid was triturated with water and dichloromethane. The title compound was dried in vacuo to afford 16 mg (2%); LCMS 3.44 min, m/z [M+H]⁺ 435.

### Example 16

### Preparation of N-(5-nitro-pyridin-2-yl)-5-Iodo-1H-indazole-3-carboxamide

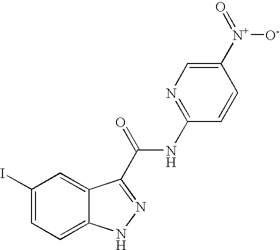

Procedure B was followed using the amide produced in Example 16A. Water and dichloromethane were removed by filtration and the solid was triturated with water and dichloromethane. The title compound was dried in vacuo to afford 5 mg (1%); LCMS 4.50 min, m/z [M+H]⁺ 410.

### Example 17

### Preparation of 5-Morpholin-4-yl-1H-indazole-3-carboxylic acid phenylamide

### 17A. Preparation of 5-Nitro-1H-indazole-3-carboxylic acid

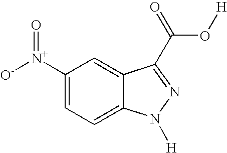

To a suspension of indazole-3-carboxylic acid (Fluka) (5 g, 31 mmol) in concentrated H₂SO₄ (30 ml) at 0° C. was added KNO₃ (3.13 g, 31 mmol). The reaction was allowed to stir overnight at room temperature, then diluted with water and the products extracted with ethyl acetate. The combined organic layers were washed with brine and then dried over MgSO₄. Evaporation to dryness left the product as a yellow solid as a 7:3 mixture with the 7-nitro isomer; LCMS 2.58 min, m/z [M+H]⁺ 208.

### 17B. Preparation of 5-Nitro-1H-indazole-3-carboxylic acid methyl ester

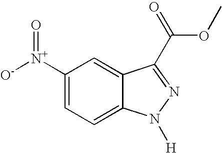

To a suspension of the carboxylic acid 1A (2.5 g, 12.1 mmol) in methanol (40 ml) was added concentrated hydrochloric acid (3 drops). The reaction was heated to reflux overnight. The reaction was allowed to cool to room temperature. The solid was filtered and dried in a vacuum oven to leave a yellow solid; LCMS 3.30 min, m/z [M+H]⁺ 222 and m/z [2M+H]⁺ 443.

### 17C. Preparation of 5-Amino-1H-indazole-3-carboxylic acid methyl ester

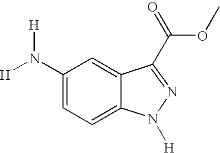

To a suspension of the nitro-indazole 1B (1.23 g, 5.57 mmol) in ethanol (10 ml) was added ethyl acetate (50 ml) and then Pd/C (56 mg) under a nitrogen atmosphere. The atmosphere was exchanged for H₂, and H₂ was bubbled through the reaction mixture for 5 minutes. After three hours the compound was observed to have dissolved completely. The reaction mixture was filtered though Celite and the filtrate evaporated to dryness to leave the product amine [which contains approximately 25% of the 7-nitro isomer] as a yellow solid; LCMS 2.68 min, [M+H]⁺ 192.

### 17D. Preparation of 5-morpholin-4-yl-1H-indazole-3-carboxylic acid methyl ester

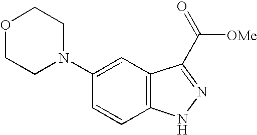

To a mixture of 5-amino-1H-indazole-3-carboxylic acid methyl ester and 7-amino-1H-indazole-3-carboxylic acid methyl ester (as synthesized above) (1.91 g, 10.0 mmol, 1.0 equiv) in DMF (20 ml) was added N,N-diisopropylethylamine (5.2 ml, 30.0 mmo, 3.0 equiv), tetrabutylammonium iodide (739 mg, 2.0 mmol, 0.2 equiv) and bis(chloroethyl)ether (1.4 ml, 12.0 mmol, 1.2 equiv). The solution was heated to 90° C. for 15 h. The DMF was carefully removed under reduced pressure in a fume hood. The resultant mixture was partitioned between ethyl acetate and water. The organic layer was washed with brine, dried (MgSO₄), and concentrated under reduced pressure. The compound was purified by column chromatography to afford 5-morpholin-4-yl-1H-indazole-3-carboxylic acid methyl ester 300 mg (11%); LCMS 2.28 min, m/z [M+H]⁺ 262.

### 17E. Preparation of 5-Morpholin-4-yl-1H-indazole-3-carboxylic acid phenylamide

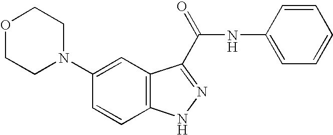

To 5-morpholin-4-yl-1H-indazole-3-carboxylic acid methyl ester (91 mg, 0.35 mmol, 1.0 equiv) in THF (3 ml) was added potassium hydroxide (116 mg, 1.75 mmol, 5.0 equiv) in water (3.5 ml). The mixture was heated to reflux for 3.5 h. The mixture was evaporated to dryness and 2N hydrochloric acid was added. The resultant precipitate was collected and azeotroped with toluene (3×10 ml).

The crude 5-Morpholin-4-yl-1H-indazole-3-carboxylic acid solid LCMS 1.78 min, m/z [M+H]⁺ 248 was used directly in Procedure A. The aqueous was extracted with dichloromethane. The combined organic layers were washed with brine, dried (MgSO₄) and were removed under reduced pressure. The title compound was further purified by preparative HPLC to afford 9 mg (16%); LCMS 3.11 min, m/z [M+H]⁺ 323.

### Example 18

### Preparation of 5-Nitro-1H-indazole-3-carboxylic acid (4-sulphamoyl-phenyl)-amide

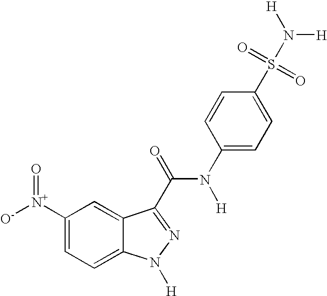

Procedure B was followed using 5-Nitro-1H-indazole-3-carboxylic acid (Example 17A) and 4-amino-benzenesulphonamide. Water and dichloromethane were removed by filtration and the solid was triturated with water and dichloromethane. The title compound was further purified by preparative HPLC as a 8:2 mixture with the 7-nitro isomer; LCMS 2.89 min, m/z [M+H]⁺ 362.

### Example 19

### Preparation of 5-Nitro-1H-indazole-3-carboxylic acid (4-methylsulphamoylmethyl-phenyl)-amide

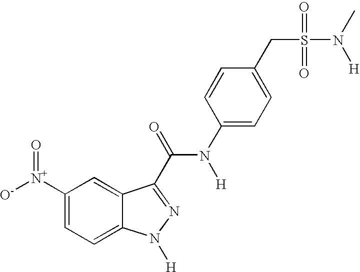

Procedure B was followed using 5-Nitro-1H-indazole-3-carboxylic acid (Example 17A) and (4-amino-phenyl)-N-methyl-methane sulphonamide. Water and dichloromethane were removed by filtration and the solid was triturated with water and dichloromethane. The title compound was further purified by preparative HPLC: LCMS 3.30 min, m/z [M+H]⁺ 390.

### Example 20

### General Palladium (0) Cross-Coupling Procedure C

To 5-iodo-1H-indazole-3-carboxylic acid (4-methylsulphamoylmethyl-phenyl)-amide (Example 13) (47 mg, 0.1 mmol, 1.0 equiv.) in toluene (0.8 ml) was added the relevant palladium (0) catalyst (0.02 mmol, 0.2 equiv.). The reaction mixture was degassed by bubbling nitrogen through the mixture and was stirred at room temperature for 5 minutes. The corresponding heteroaryl boronic acid (0.3 mmol, 3.0 equiv) in ethanol (0.8 ml) was added and stirred for 5 minutes. To the mixture was added a solution of potassium carbonate (138 mg, 1.0 mmol, 10 equiv.) in water (2.0 ml) followed by methanol (2.0 ml) and the mixture was sealed in a vial under nitrogen. The mixture was heated between 120° C. and 150° C. for 15 minutes using a maximum 100-watt power in a microwave. Methanol (5 ml) was added and all solvents were removed under reduced pressure. The compounds were purified as described in the Examples below, and characterised by liquid chromatography and mass spectrometry using either of the systems described above.

### Example 21

### Preparation of 5-Thiophen-2-yl-1H-indazole-3-carboxylic acid (4-methylsulphamoylmethyl-phenyl)-amide

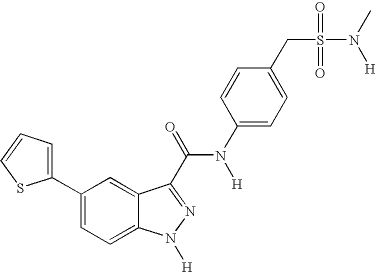

Procedure C was followed using bis(tri-t-butylphosphine)palladium (0) (Strem) and thiophene-2-boronic acid (Maybridge). The solid was triturated with water. The title compound was further purified by preparative HPLC to afford 22 mg (52%); LCMS 3.97 min, m/z [M+H]⁺ 427.

### Example 22

### Preparation of 5-(3,5-Dimethyl-isoxazol-4-yl)-1H-indazole-3-carboxylic acid (4-methylsulphamoylmethyl-phenyl)-amide

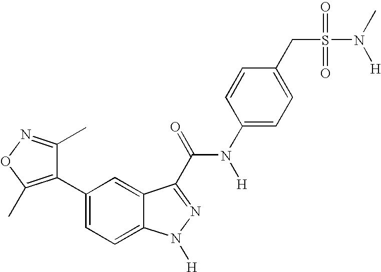

Procedure C was followed using bis(tri-t-butylphosphine)palladium (0) (Strem) and 3,5-dimethylisoxazole-4-boronic acid (Maybridge). The solid was triturated with water. The title compound was further purified by preparative HPLC to afford 5 mg (11%); LCMS 3.54 min, m/z [M+H]⁺ 440.

### Example 23

### Preparation of 5-Furan-2-yl-1H-indazole-3-carboxylic acid (4-methylsulphamoylmethyl-phenyl)-amide

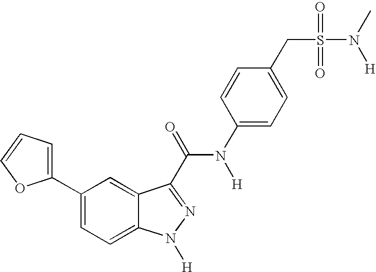

Procedure C was followed using bis(tri-t-butylphosphine)palladium (0) (Strem) and furan-2-boronic acid (Lancaster). The solid was triturated with water. The title compound was further purified by preparative HPLC to afford 15 mg (37%): LCMS 3.82 min, m/z [M+H]⁺ 411.

### Example 24

### Preparation of 5-Benzofuran-2-yl-1H-indazole-3-carboxylic acid (4-methylsulphamoylmethyl-phenyl)-amide

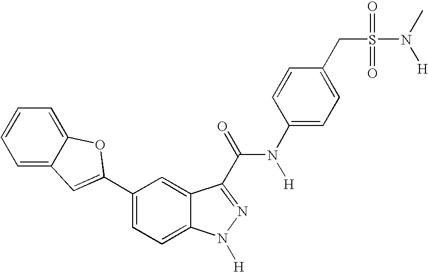

Procedure C was followed using tetrakis(triphenylphosphine)palladium(0) (Aldrich) and benzo[b]furan-2-boronic acid (Lancaster). The solid was triturated with water. The title compound was further purified by preparative HPLC to afford 20 mg (36%): LCMS 4.33 min, m/z [M+H]⁺ 461.

### Example 25

### Preparation of 5-Chloro-1H-indazole-3-carboxylic acid (4-methylsulphamoylmethyl-phenyl)-amide

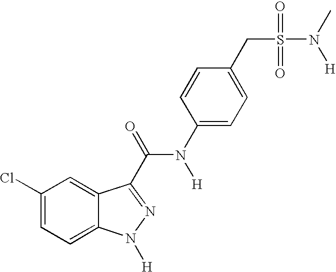

To a solution of 5-iodo-1H-indazole-3-carboxylic acid (4-methylsulphamoyl-methyl-phenyl)-amide (Example 13) (42 mg, 0.09 mmol, 1.0 equiv.) in d6-dimethyl sulphoxide (0.7 ml) was added copper(I) chloride (401 mg, 4.05 mmol, 45 equiv.). The mixture was heated to 180° C. for 15 minutes using a maximum 50-watt power in a microwave. The title compound was purified by preparative HPLC to afford 14 mg (41%); LCMS 3.54 min, m/z [M+H]⁺ 379.

### Example 26

### Preparation of 1H-Indazole-3-carboxylic acid (4-sulphamoyl-phenyl)-amide

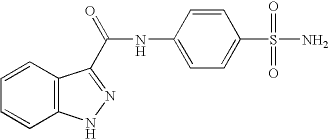

To indazole-3-carboxylic acid (1 equiv.) in N-methylpyrrolidinone (5 ml) was added EDC (1.2 equiv.), HOBT (1.2 equiv.), NMM (1.2 equiv.) and then 4-sulphamoyl aniline (1.3 equiv.) at room temperature. The reaction was heated to 100° C. for 24 hours. A further equivalent of EDC was added and the reaction heated at 100° C. for a further 4 hours. Water was added to the reaction and the aqueous layer extracted with ethyl acetate (2×30 ml). The combined organic layers were dried (MgSO₄) and the solvent removed under reduced pressure. The desired product was isolated by column chromatography. LCMS 2.72 min, m/z [M+H]⁺ 317.

### Example 27

### Preparation of 1H-Indazole-3-carboxylic acid phenyl)-amide

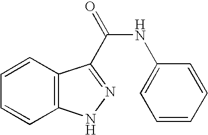

By following the procedure described in Example 26, but using aniline instead of 4-sulphamoyl aniline, the title compound was prepared. LCMS 3.44 min, m/z [M+H]⁺ 238.

### Example 28

### 1H-Indazole-3-carboxylic acid (6-chloro-pyridin-3-yl)-amide

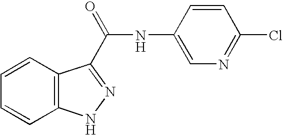

By following procedure A, the title compound was prepared; LCMS m/z [M+H]⁺ 273, 3.42 min.

### Example 29

### Preparation of 5-Thiazol-2-yl-1H-indazole-3-carboxylic acid (4-methylsulphamoylmethyl-phenyl)-amide

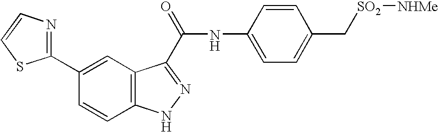

A solution of 5-iodo-1H-indazole-3-carboxylic acid (4-methylsulphamoylmethyl-phenyl)-amide (Example 13) (47 mg, 0.1 mmol, 1.0 equiv.) in THF (1 ml) was degassed by bubbling nitrogen though the solution. Bis(tri-tert-butylphosphine)palladium(0) (23 mg, 0.02 mmol, 0.2 equiv.) was added, the solution was degassed with nitrogen and stirred for 5 minutes. 2-Thiazolylzinc bromide (2 ml of a 0.5M solution in THF, 1.0 mmol, 10 equiv) was added and the mixture was heated to 195° C. for 15 minutes using 100 watts in a CEM microwave. The reaction was quenched with methanol and evaporated to dryness.

The title compound was purified by Biotage (SiO₂), eluted with 80% EtOAc-petrol, to afford 18 mg (42%); LCMS 3.27 min, m/z [M+H]⁺ 428.

### Examples 30-59

By following procedures A, B or C as set out above, and using the appropriate starting materials, the compounds set out in Table 1 below were prepared.

TABLE 1

| | | | m/z [M + H]⁺ |
|---|---|---|---|
| EXAMPLE | PROCEDURE | COMPOUND | LCMS (min) |
| 30 | B | 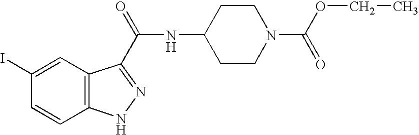 | 371 |
| 31 | A | 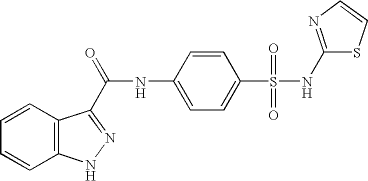 | 400, 3.09 |
| 32 | C | 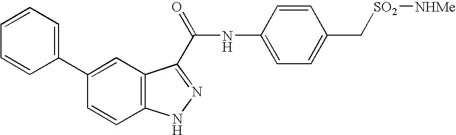 | 421, 3.88 min |
| 33 | A | 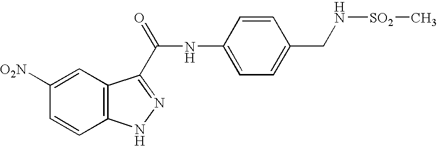 | 390 3.07 min |
| 34 | A | 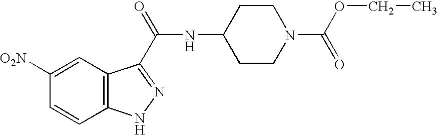 | 362 3.17 min |
| 35 | A | 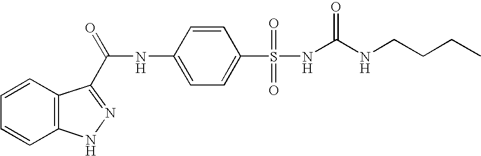 | 416 |
| 36 | A | 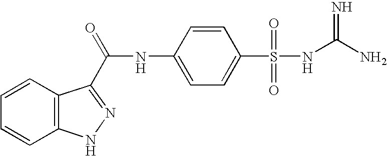 | 359 |
| 37 | B | 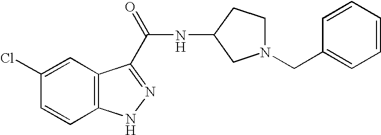 | 355, 2.29 min |
| 38 | B | 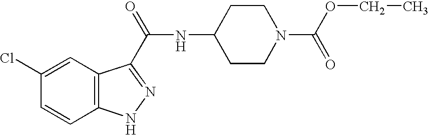 | 351, 3.42 min |
| 39 | B | 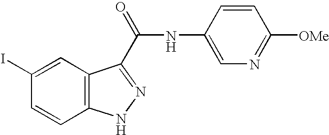 | 395, 3.84 min |
| 40 | B | 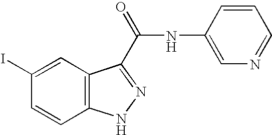 | 365, 2.56 min |
| 41 | B | 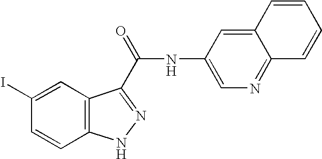 | 415, 3.88 min |
| 42 | B | 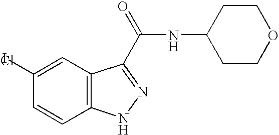 | 280, 2.93 min |
| 43 | B | 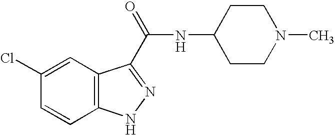 | 293, 1.91 min |
| 44 | B | 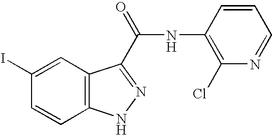 | 399, 4.30 min |
| 45 | B | 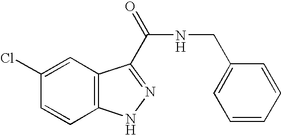 | 286, 3.79 min |
| 46 | B | 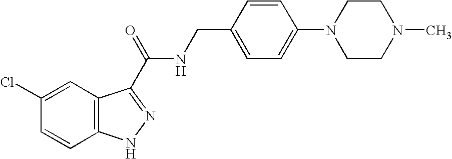 | 384/386 2.30 min |
| 47 | B | 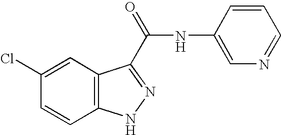 | 273, 2.36 min |
| 48 | B | 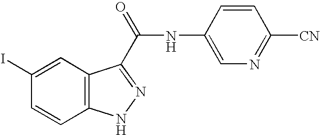 | 390, 3.97 min |
| 49 | B | 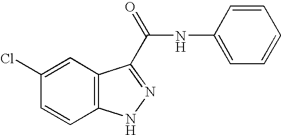 | 272, 4.02 min |
| 50 | B | 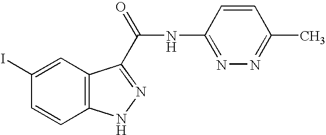 | 380, 3.41 min |
| 51 | B | 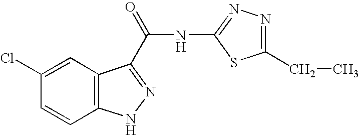 | 308, 3.62 min |
| 52 | B | 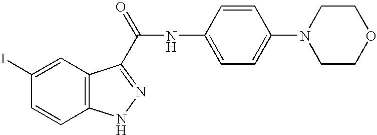 | 449, 3.69 min |
| 53 | B | 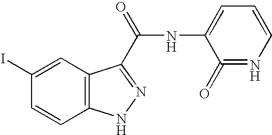 | 381, 3.52 min |
| 54 | A | 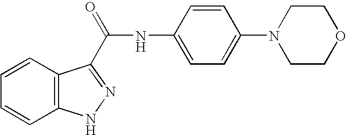 | 323, 2.93 min |
| 55 | A | 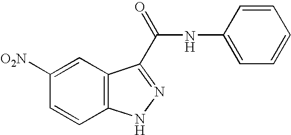 | 283, 3.91 min |
| 56 | B | 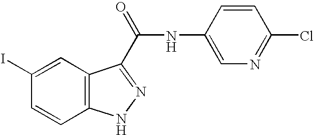 | 399, 4.42 min |
| 57 | A | 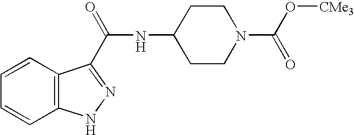 | 345, 3.65 min |
| 58 | B | 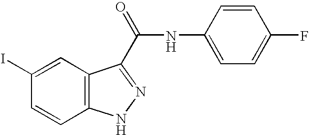 | 381 |
| 59 | B | 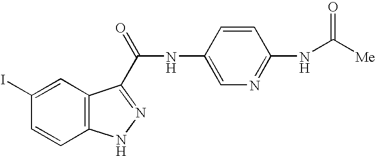 | 421 |

### Example 60

### 5-Amino-1H-indazole-3-carboxylic acid phenylamide

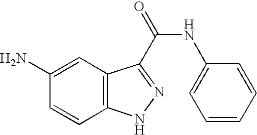

To a suspension of the nitro-indazole of Example 55 (49 mg, 0.17 mmol) in ethanol (5 ml) was added Pd/C (0.1 equiv.) under a nitrogen atmosphere. The atmosphere was exchanged for H₂, and H₂ was bubbled through the reaction mixture for 5 minutes. The reaction was left for 16 hours and flushed with N₂, following which the reaction mixture was filtered though Celite and the filtrate evaporated to dryness to leave the product amine as a red-brown solid. LCMS 2.09 min m/z [M+H]⁺ 253.

### Example 61

### 5-Iodo-1H-indazole-3-carboxylic acid (4-methylaminosulphonylmethyl-phenyl)-amide

### 61A. (4-methylaminosulphonylmethyl-phenyl)-amine

To aminobenzylamine (1 g, 8.18 mmol) in CH₂Cl₂ (50 ml) at 0° C. was added Et₃N (2.28 ml, 16.3 mmol) followed by MesCl (0.63 ml, 8.18 ml), and the reaction was stirred at 0° C. for 1 hour. The reaction mixture was diluted with CH₂Cl₂, and washed twice with water. The combined organic layers were dried, filtered and evaporated to dryness. The product was purified by trituration with 5% MeOH—CH₂Cl₂.

### 61B. 5-Iodo-1H-indazole-3-carboxylic acid (4-methylaminosulphonylmethyl-phenyl)-amide

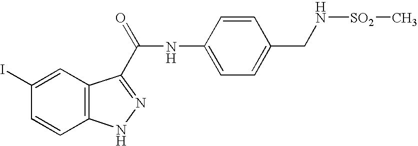

The product of Example 61A was reacted with 5-iodo indazole-3-carboxylic acid using method B to give the title compound. LCMS 3.66 min m/z [M+H]⁺ 471.

### Example 62

### 62A. 5-Amino-1H-indazole-3-carboxylic acid (4-sulphamoyl-phenyl)-amide

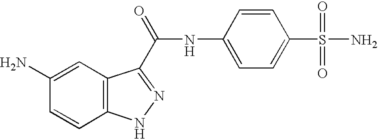

To a suspension of the nitro-indazole of Example 18 (1.0 g, 2.77 mmol) in DMF:EtOH (1:1, 20 ml) was added Pd/C (0.27 mg, 0.1 eq) under a nitrogen atmosphere. The atmosphere was exchanged for H₂, and H₂ was bubbled through the reaction mixture for 5 minutes. After three hours the compound was observed to have dissolved completely. The reaction mixture was filtered though Celite and the filtrate evaporated to dryness to leave the product amine. Purification by preparative HPLC gave the desired product. LCMS 0.58 min m/z [M+H]⁺ 332.

### 62B. 7-Amino-1H-indazole-3-carboxylic acid (4-sulphamoyl-phenyl)-amide

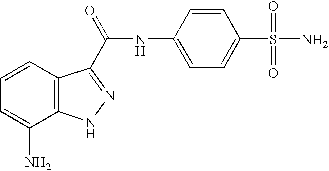

The 7-amino isomer was isolated as a minor product the reaction described in Example 62A. LCMS 2.32 min m/z [M+H]⁺ 332.

### Example 63

### 5-[3-(2-Chloro-ethyl)-ureido]-1H-indazole-3-carboxylic acid (4-methylsulphamoylmethyl-phenyl)-amide

### 63A. Preparation of 5-Nitro-1H-indazole-3-carboxylic acid

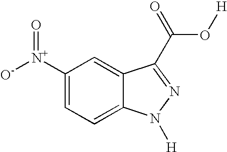

To a suspension of indazole-3-carboxylic acid (Fluka) (5 g, 3 mmol) in concentrated H₂SO₄ (30 ml) at 0° C. was added KNO₃ (3.13 g, 31 mmol). The reaction was allowed to stir overnight at room temperature, then diluted with water and the products were extracted with ethyl acetate. The combined organic layers were washed with brine and then dried over MgSO₄. Evaporation to dryness left the product as a yellow solid as a 7:3 mixture with the 7-nitro isomer; LCMS 2.58 min, m/z [M+H]⁺ 208.

### 63B. Preparation of 5-Nitro-1H-indazole-3-carboxylic acid (4-methylsulphamoylmethyl-phenyl)-amide

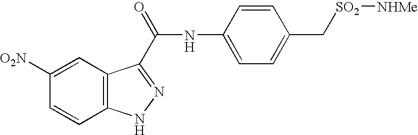

To the nitro-1H-indazole-3-carboxylic acid (1 equiv.) of Example 63A in DMF (0.3 M) was added EDC (1.2 equiv.), HOBT (1.2 equiv.), NMM (1.2 equiv.) and then 4-methylsulphamoylmethyl-phenylamine (1.3 equiv.) at room temperature. The reaction was heated to 70° C. for 2 hours and then stirred at room temperature for 48 hours. Water was added to the reaction mixture and the precipitated product was filtered. The solid was washed with water, then a small volume of MeOH, and then dried in a vacuum oven to leave a yellow solid.

### 63C. Preparation of 5-Amino-1H-indazole-3-carboxylic acid (4-methylsulphamoylmethyl-phenyl)-amide

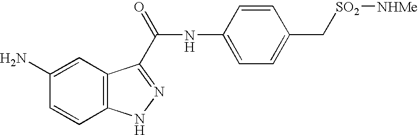

To a suspension of the resulting nitro-indazole (1.0 g, 2.57 mmol) in ethanol: DMF (1:1, 20 ml) was added Pd/C (0.1 equiv.) under a nitrogen atmosphere. The atmosphere was exchanged for H₂, and H₂ was bubbled through the reaction mixture for 5 minutes. After three hours the compound was observed to have dissolved completely. The reaction mixture was filtered though Celite and the filtrate evaporated to dryness to leave the product amine as a brown solid.

### 63D. 5-[3-(2-Chloro-ethyl)-ureido]-1H-indazole-3-carboxylic acid (4-methylsulphamoylmethyl-phenyl)-amide

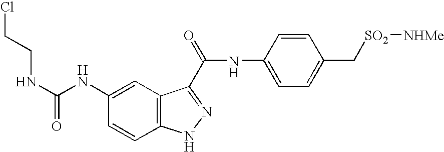

To a suspension of the amine (0.28 mmol) in THF (1 ml) at room temperature was added 2-chloroethyl isocyanate (0.42 mmol, 1.5 eq). The reaction was heated to 70° C. for 4 hours. The colour of the suspension changed from light brown to a much darker brown. Water (10 ml) was added to quench the reaction and the precipitate was filtered. The solid was washed with a portion of water and the THF and dried to leave a grey product. LCMS 2.88 min m/z [M+H]⁺ 465/467.

### Example 64

### 5-Iodo-1H-indazole-3-carboxylic acid piperidin-4-ylamide

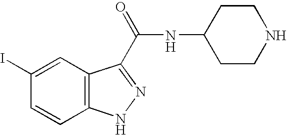

To a solution of the compound of Example 30 (0.16 g, 0.36 mmol) at 0° C., in a mixture of THF: H₂O (9.5 ml: 4 ml) was added LiOH (30 mg, 0.72 mmol) followed by MeOH (4 ml). The reaction was stirred at room temperature, and when no reaction occurred the total LiOH added was increased to 150 mg. The reaction mixture was heated at 60° C. for 8 hours, and then evaporated to dryness. The product was purified by preparative HPLC to afford 40 mg, m/z [M+H]⁺ 371.

### Example 65

### 5-Chloro-1H-indazole-3-carboxylic acid [4-(acetylamino-methyl)-phenyl]-amide

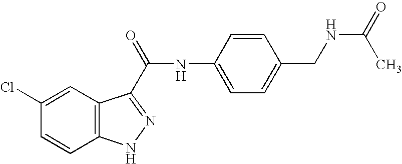

N-(4-Amino-benzyl)-acetamide produced by the method of Example 15A was reacted with 5-chloro-1H-indazole-3-carboxylic acid following procedure B to give the title compound. LCMS 3.90 min m/z [M+H]⁺ 343.

### Example 66

### Preparation of 1H-Indazole-3-carboxylic acid [1-(2,2,2 trifluoro-acetyl)-Piperidin-4-yl]-amide

### 66A. 1H-Indazole-3-carboxylic acid piperidin-4-ylamide.TFA salt

To a suspension of the compound of Example 57 (0.4 g, 1.16 mmol) in DCM (30 ml) at 0° C. was added TFA (3 ml), and the reaction was stirred at room temperature for 1 hour. The mixture was evaporated down, and then azeotroped with toluene to dryness. The solid was triturated with ether to afford the title compound (0.3 g).

### 66B. 1H-Indazole-3-carboxylic acid [1-(2,2,2 trifluoro-acetyl)-pipieridin-4-yl-amide

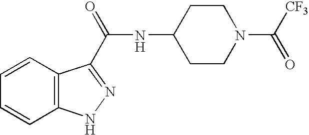

To a suspension of 1H-indazole-3-carboxylic acid piperidin-4-ylamide.TFA salt, (the product of 66A) (50 mg, 0.2 mmol) in dichloromethane (0.5 ml) and pyridine (0.5 ml) at 0° C. was added dropwise methanesulphonic anhydride (0.2 mmol), and the mixture was allowed to warm up to room temperature. The reaction mixture was diluted with water and washed with ethyl acetate. The combined organic layers were dried (MgSO₄), filtered and evaporated to dryness to give a yellow oil. The title compound was purified by column chromatography, by elution with 2% MeOH/EtOAc then 5% MeOH/EtOAc, to afford 28 mg of the title compound. LCMS 3.34 min, m/z [M+H]⁺ 341.

### Example 67

### Preparation of 1H-Indazole-3-carboxylic acid piperidin-4-ylamide

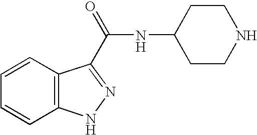

To a suspension of the compound of Example 57 (0.4 g, 1.16 mmol) in CH₂Cl₂ (10 ml) at 0° C. was added TFA (3 ml), and the reaction was stirred at room temperature for 1 hour. The reaction mixture was evaporated to dryness and then azeotroped with toluene. The product was triturated with ether. The sample was neutralised, and then purified by preparative HPLC to afford the purified product 8 mg. m/z [M+H]⁺ 245

### Example 68

### 1H-Indazole-3-carboxylic acid (1-acetyl-piperidin-4-yl)-amide

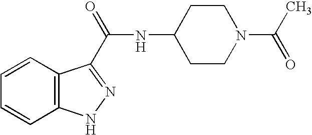

To a suspension of the compound of Example 67 (50 mg, 0.2 mmol) in CH₂Cl₂ (0.5 ml) and pyridine (0.5 ml) at 0° C. was added acetic anhydride (0.22 mmol) dropwise, and the reaction was allowed to warm up to room temperature. The reaction mixture was diluted with water, and washed with ethyl acetate. The combined organic layers were dried, filtered and evaporated to give a yellow oil. Column chromatography using 5% MeOH/CH₂Cl₂ then 7% MeOH/CH₂Cl₂ afforded 20 mg of product, m/z [M+H]⁺ 287.

### Example 69

### 1H-Indazole-3-carboxylic acid (1-methanesulphonyl-piperidin-4-yl)-amide

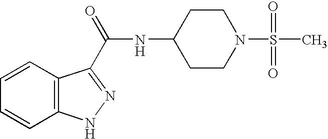

To a suspension of the compound of Example 67 (33 mg, 0.13 mmol) was added Et₃N (0.054 ml, 0.39 mmol) followed by THF (0.5 ml), DMSO (0.5 ml) and then methanesulphonyl chloride (0.01 ml, 0.13 mmol). The reaction was stirred at room temperature overnight. The reaction mixture was reduced by evaporation, and purified by preparative HPLC to afford 10 mgs of the product, m/z [M+H]⁺ 323.

### Example 70

### 1H-Indazole-3-carboxylic acid (4-fluoro-phenyl)-amide

### 70A. N-(4-Fluoro-phenyl)-2-(2-nitro-phenyl)-acetamide

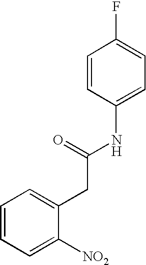

To (2-Nitro-phenyl)-acetic acid (1 equiv.) in DCM (0.3 M) was added EDC (2 equiv.), HOBT (2 equiv.), NMM (2 equiv.) and then corresponding amine (1.5 equiv.) at room temperature. The reaction was left at room temperature for 5 hours. The reaction was diluted with water and extracted with DCM (×3). The combined organic layers were washed with brine and dried over MgSO₄. The product was filtered and evaporated to dryness to leave a yellow solid, which was taken onto the next reaction; LCMS MH⁺ 275, RT 3.57 min.

### 70B. 2-(2-Amino-phenyl)-N-(4-fluoro-phenyl)-acetamide

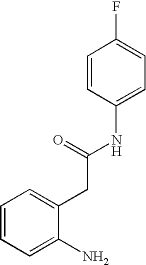

To a suspension of the nitro compound (7 g, 25.5 mmol) in EtOH (225 ml) was added Pd/C (0.1 eq) under a nitrogen atmosphere. The atmosphere was exchanged for H₂, and H₂ was bubbled through the reaction mixture for 5 minutes. After 48 hours the reaction mixture was filtered though Celite and the filtrate evaporated to dryness to leave the product amine, which was taken on to the next reaction; LCMS MH⁺ 245, RT 2.57 min.

### 70C. 1H-Indazole-3-carboxylic acid (4-fluoro-phenyl)-amide

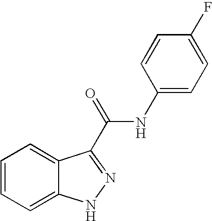

To a solution of the amine (3.0 g, 12.2 mmol) in toluene (122 ml) was added acetic anhydride (3.9 ml, 40.5 mmol) at room temperature. The reaction was heated to 90-95° C. To this mixture was added isopentyl nitrate (3.4 ml, 24.6 mmol) dropwise over a period of about 20 minutes, at 90-95° C. The mixture was left for 90 minutes, and then heated to 105° C. for 16 hours. The reaction had turned from a yellow to a red suspension. The reaction was evaporated to dryness and then taken up in EtOAc and washed with water. The organic layer was extracted with brine and dried over MgSO₄. The product was filtered and evaporated to dryness in vacuo to leave an oil which was purified by HPLC; LCMS MH⁺ 2.56, RT 3.69 min.

### Example 71

### 4-Bromo-1H-indazole-3-carboxylic acid (4-fluoro-phenyl)-amide

### 71A. Preparation of 5-nitro-1H-indazole-3-carboxylic acid (4-fluorophenyl)-amide

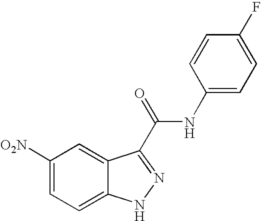

To a solution of 5-nitro-1H-indazole-3-carboxylic acid (Example 17A) (6.5 g, 31.5 mmol, 1.0 equiv) in DMF (200 ml) was added 4-fluoroaniline (33.3 ml 34.6 mmol, 1.1 equiv), HOBt (5.1 g, 37.7 mmol, 1.2 equiv) and EDC (7.2 g, 37.7 mmol, 1.2 equiv). The mixture was stirred for a period of 72 hours. The solvent was removed under reduced pressure and the resulting solid suspended in ethyl acetate and aqueous sodium hydrogen carbonate. The precipitate was collected, resuspended in aqueous sodium hydrogen carbonate and stirred for 10 mins. The solid was collected and dried in a vacuum oven to afford the title compound (7.77 g, 82%) as a 8:2 mixture with the 7-nitro isomer; LCMS 3.83 min, m/z [M+H]⁺ 300.

### 71B. Preparation of 5-amino-1H-indazole-3-carboxylic acid (4-fluorophenyl)-amide

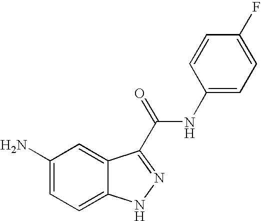

A mixture of 5-nitro-1H-indazole-3-carboxylic acid (4-fluorophenyl)-amide (7.3 g, 24.3 mmol), 10% Pd/C (0.7 g), ethanol (200 ml) and DMF (200 ml) under an atmosphere of nitrogen was stirred under an atmosphere of hydrogen for 18 hours. Then the catalyst was removed and the filtrate was evaporated to dryness, to give the title compound (4.94 g, 75%) as a 8:2 mixture with the 7-nitro isomer; LCMS 1.95 min, m/z [M+H]⁺ 270.

### 71C. Preparation of 5-Amino-4-bromo-1H-indazole-3-carboxylic acid (4-fluorophenyl)-amide

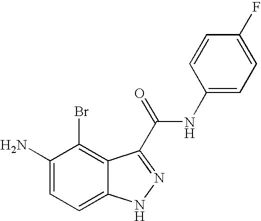

Bromine was added dropwise to a stirred suspension of 5-amino-1H-indazole-3-carboxylic acid (4-fluorophenyl)-amide (4.9 g, 18.3 mmol) in MeOH (10.5 ml) at −5° C. The reaction mixture was stirred at −5° C. for 1 hour, and then allowed to warm to 10° C. The reaction was poured into aqueous sodium thiosulphate solution and the suspension was stirred. The solid was collected, washed with water and then dried in a vacuum oven to afford the title compound 32C (6.9 g) that was used without further purification: LCMS 2.89 min, m/z [M+H]⁺ 348.

### 71D. Preparation of 4-bromo-1H-indazole-3-carboxylic acid (4-fluorophenyl)-amide

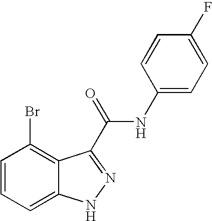

To a solution of the 5-amino-4-bromo-1H-indazole-3-carboxylic acid (4-fluorophenyl)-amide (1.5 g, 4.2 mmol) in DMF (14 ml) was added the isopentyl nitrate (0.89 ml, 6.4 mmol) slowly at 65° C. After 5 minutes, effervescence was noted. The reaction left for a further 2 hours and allowed to cool. HCl (1 M, aq.) was added to the reaction and the product was filtered off. The solid was washed with water and evaporated down from toluene (×2). The compound was purified by prep HPLC; LCMS MH⁺ 334/336, RT 3.65 min.

### Example 72

### 5-Methyl-1H-indazole-3-carboxylic acid (4-fluoro-phenyl)-amide

### 72A. Preparation of 5-methyl-1H-indazole-3-carboxylic acid

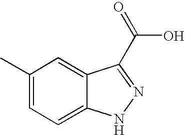

To a suspension of 5-methylisatin (Lancaster Synthesis) (5.8 g, 36.0 mmol) in water (90 ml) was added NaOH (1.53 g, 38.2 mmol, 1.1 equiv) and the mixture was warmed to approximately 35° C. for 30 minutes to form a solution. The solution was cooled to 5° C. and a solution of sodium nitrite (2.78 g, 40.3 mmol, 1.1 equiv) was added dropwise over approximately 30 minutes, keeping the temperature below 10° C. The whole mixture was added dropwise via a cannula to a vigorously stirred solution of concentrated sulphuric acid (7.3 g, 74.4 mmol, 2.0 equiv) in water (90 ml) keeping the temperature below 10° C. The mixture was stirred for 20 minutes and a solution of tin (II) chloride (16.7 g, 74.4 mmol, 2.4 equiv) in concentrated hydrochloric acid (34 ml) was added dropwise. The mixture was stirred at 5° C. for 2 hours and the resulting crude 5-methylindazole-3-carboxylic acid was isolated by filtration and washed several times with water. The yellow solid was then azeotroped with toluene (3×100 ml) to remove water prior to the next step to leave a yellow/green solid. LCMS MH⁺ 177, RT 2.40 min.

### 72B. 5-Methyl-1H-indazole-3-carboxylic acid (4-fluoro-phenyl)-amide

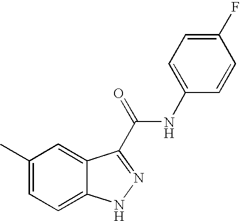

To the carboxylic acid (1 equiv.) in DCM (0.3 M) was added EDC (1.2 equiv.), HOAT (1.2 equiv.), and then corresponding amine (1.3 equiv.) at room temperature. The reaction was left at room temperature for 48 hours. The reaction was diluted with water and extracted with EtOAc (×3). The combined organic layers were washed with brine and dried over MgSO₄. The product was filtered and evaporated to dryness to leave a yellow solid. The product was triturated with DCM to yield the product; MH⁺ 270, RT 4.08 min.

### Example 73

### 6-Bromo-1H-indazole-3-carboxylic acid (4-fluoro-phenyl)-amide

### 73A. Preparation of 6-bromo-1H-indazole-3-carboxylic acid

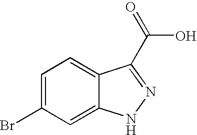

To a suspension of 6-bromoisatin (Richman) (5.0 g, 22.1 mmol) in water (55 ml) was added NaOH (0.94 g, 23.5 mmol, 1.1 equiv) and the mixture was warmed to approximately 35° C. for 30 minutes to form a solution. The solution was cooled to 5° C. and a solution of sodium nitrite (1.70 g, 24.8 mmol, 1.1 equiv) was added dropwise over approximately 30 minutes, keeping the temperature below 10° C. The whole mixture was added dropwise via a cannula to a vigorously stirred solution of concentrated sulphuric acid (4.48 g, 45.7 mmol, 2.0 equiv) in water (55 ml) keeping the temperature below 10° C. The mixture was stirred for 20 minutes and a solution of tin (II) chloride (10.2 g, 54.0 mmol, 2.4 equiv) in concentrated hydrochloric acid (21 ml) was added dropwise. The mixture was stirred at 5° C. for 2 hours and the resulting crude 5-methylindazole-3-carboxylic acid was isolated by filtration and washed several times with water. The yellow solid was then azeotroped with toluene (3×100 ml) to remove water prior to the next step to leave a yellow/green solid. LCMS MH⁺ 238/240 (⁷⁹Br/⁸¹Br), RT 2.69 min.

### 73B. Preparation of 6-Bromo-1H-indazole-3-carboxylic acid (4-fluoro-phenyl)-amide

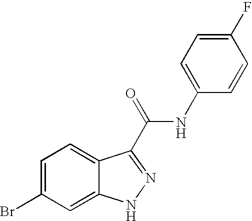

To 6-bromo-1H-indazole-3-carboxylic acid (1 equiv.) in DCM (0.3 M) was added EDC (1.2 equiv.), HOAT (1.2 equiv.), and then corresponding amine (1.3 equiv.) at room temperature. The reaction was left at RT for 4 hours. The reaction was diluted with water and extracted with EtOAc (×2). The combined organic layers were washed with brine and dried over MgSO₄. The product was filtered and evaporated to dryness to leave a yellow solid. The product was triturated with DCM, and purified further by prep HPLC; MH⁺ 334/336 (⁷⁹Br/⁸¹Br), RT 4.32 min.

### Examples 74-80

By following the procedures described in the examples above, and using the appropriate starting materials, the compounds set out in Table 2 below were prepared.

TABLE 2

| EXAMPLE | PROCEDURE | COMPOUND | m/z [M + H]⁺ |
|---|---|---|---|
| 74 | B | 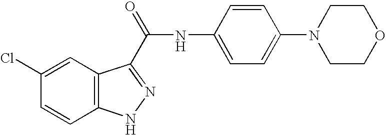 | 357, RT 3.51 min |
| 75 | B | 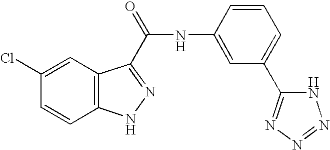 | 340, RT 3.39 min |
| 76 | B | 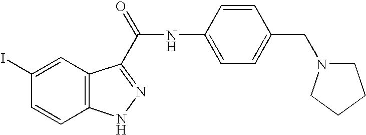 | 355, RT 3.56 min |
| 77 | B | 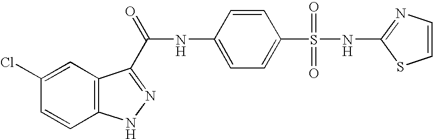 | 434, RT 3.37 min |
| 78 | A | 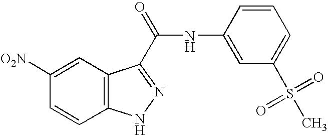 | 361, RT 3.58 min |
| 79 | A | 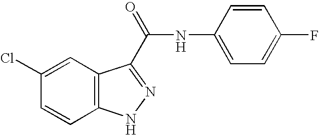 | 290/292, RT 4.11 min |
| 80 | B | 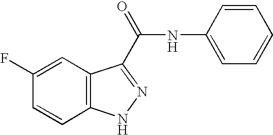 | 256 |

### Example 81

### Preparation of 3-[(5-Chloro-1H-indazole-3-carbonyl)-amino]-pyrrolidine-1-carboxylic acid methyl ester

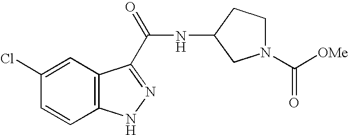

To a solution of 5-chloro-1H-indazole-3-carboxylic acid (1-benzyl-pyrrolidin-3-yl)-amide (Example 37) (639 mg, 1.8 mmol, 1 equiv) in dichloromethane (9 ml) was added 1-chloroethyl chloroformate (0.39 ml, 3.6 mmol, 2.0 equiv) at 0° C. The mixture was heated to reflux for 1 hour, cooled and evaporated under reduced pressure. The resultant oil was dissolved in methanol and heated at reflux for 15 hours. The solvents were removed under reduced pressure and the crude mixture was purified by preparative HPLC to afford the title compound 15 mg (3%); LCMS 2.29 min, m/z [M³⁵Cl)+H]⁺ 323.

### Example 82

### Preparation of 5-Morpholin-4-yl-1H-indazole-3-carboxylic acid (6-chloro-pyridin-3-yl)-amide

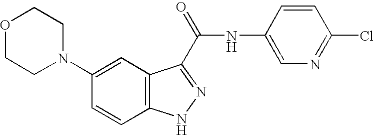

To 5-morpholin-4-yl-1H-indazole-3-carboxylic acid methyl ester (91 mg, 0.35 mmol, 1.0 equiv) (Example 17D) in THF (3 ml) was added potassium hydroxide (116 mg, 1.75 mmol, 5.0 equiv) in water (3.5 ml). The mixture was heated to reflux for 3.5 hours. The mixture was evaporated to dryness and 2N hydrochloric acid was added. The resultant precipitate was collected and azeotroped with toluene (3×10 ml).

The crude 5-Morpholin-4-yl-1H-indazole-3-carboxylic acid solid LCMS 1.78 min, m/z [M+H]⁺ 248 was used directly in Procedure A. The aqueous was extracted with dichloromethane. The combined organic layers were washed with brine, dried (MgSO₄) and were removed under reduced pressure. The title compound was further purified by preparative HPLC to afford 8 mg (13%); LCMS 3.02 min, m/z [M(³⁵Cl)+H]⁺ 358.

### Example 83

### 5-Chloro-1H-indazole-3-carboxylic acid [5-(tetrahydro-furan-2-yl)-[1.3.4]thiadiazol-2-yl]-amide

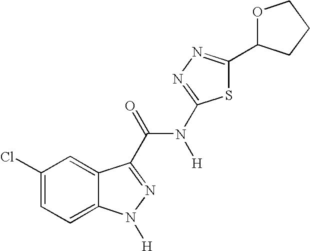

Following procedure B gave the title compound; m/z [M+H]⁺ 350.

### Example 84

### Preparation of 5-pyrrolidin-1-yl-1H-indazole-3-carboxylic acid phenylamide

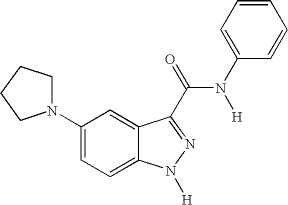

To 5-Amino-1H-indazole-3-carboxylic acid phenylamide (83 mg, 0.43 mmol, 1.0 equiv) in DMF (1.7 ml) was added N,N-diisopropylethylamine (0.23 ml, 1.30 mmol, 3.0 equiv), tetrabutylammonium iodide (32 mg, 0.09 mmol, 0.2 equiv) and 1,4-dibromobutane (0.062 ml, 0.52 mmol, 1.2 equiv). The solution was heated to 90° C. for 15 hours. The mixture was concentrated under reduced pressure and purified by preparative HPLC to afford the title compound 18 mg (14%), LCMS 3.36 min, m/z [M+H]⁺ 307.

### Example 85

### Preparation of 5-Biphenyl-2-yl-1H-indazole-3-carboxylic acid phenylamide

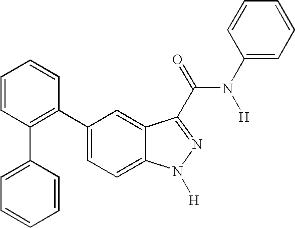

Procedure C was followed using bis(tri-t-butylphosphine)palladium (0) (Strem) and 2-biphenylboronic acid (Lancaster). The solid was triturated with water. The title compound was further purified by preparative HPLC to afford 5 mg (13%): LCMS 5.12 min, m/z [M+H]⁺ 390.

### Example 86

### Preparation of 5-(1,1-Dioxo-1lambda*6*-isothiazolidin-2-yl)-1H-indazole-3-carboxylic acid phenylamide

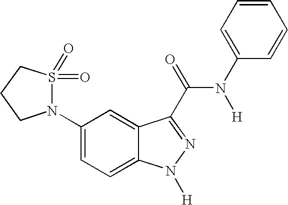

To 5-Amino-1H-indazole-3-carboxylic acid phenylamide (83 mg, 0.43 mmol, 1.0 equiv) in DMF (1.7 ml) was added N,N-diisopropylethylamine (0.23 ml, 1.30 mmol, 3.0 equiv), tetrabutylammonium iodide (32 mg, 0.09 mmol, 0.2 equiv) and 3-chloropropanesulphonyl chloride (0.092 ml, 0.52 mmol, 1.2 equiv). The solution was heated to 90° C. for 15 hours. The mixture was concentrated under reduced pressure and purified by preparative HPLC to afford the title compound 9 mg (6%), LCMS 3.32 min, m/z [M+H]⁺ 357.

### Example 87

### Preparation of 5-Phenethyl-1H-indazole-3-carboxylic acid phenylamide

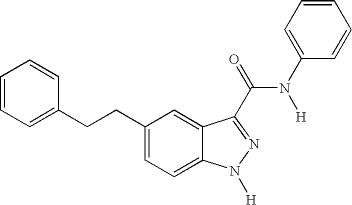

To 5-Iodo-1H-indazole-3-carboxylic acid (4-fluoro-phenyl)-amide (50 mg, 0.13 mmol, 1.0 equiv) in THF (1.3 ml) was added bis(triphenylphosphine)palladium(II) chloride (2 mg), Copper(I) iodide (1 mg), 2N NaOMe in MeOH (0.33 ml) and fluorophenylacetylene (30 mg, 0.16 mmol, 1.2 equiv). The mixture was stirred for 15 hours and concentrated under reduced pressure. 5-(6-Fluoro-3-vinyl-hepta-3,5-dien-1-ynyl)-1H-indazole-3-carboxylic acid (4-fluoro-phenyl)-amide was purified by preparative HPLC, m/z 374, 4.81 min. To 5-(6-Fluoro-3-vinyl-hepta-3,5-dien-1-ynyl)-1H-indazole-3-carboxylic acid (4-fluoro-phenyl)-amide in ethanol (13 ml) was added 10% palladium on carbon (13 mg). A hydrogen atmostphere was added and the mixture was stirred overnight. The mixture was filtered through Celite™ and concentrated under reduced pressure. The title compound was purified by preparative HPLC to afford 5 mg, m/z 342, 4.86 min.

Biological Activity

### Example 88

Measurement of CDK2 Kinase Inhibitory Activity (IC₅₀)

Compounds of the invention were tested for kinase inhibitory activity using the following protocol.

1.7 μl of active CDK2/CyclinA (Upstate Biotechnology, 10 U/μl) is diluted in assay buffer (250 μl of 10× strength assay buffer (200 mM MOPS pH 7.2, 250 mM β-glycerophosphate, 50 mM EDTA, 150 mM MgCl₂), 11.27 μl 10 mM ATP, 2.5 μl 1M DTT, 25 μl 100 mM sodium orthovanadate, 708.53 μl H₂O), and 10 μl mixed with 10 μl of histone substrate mix (60 μl bovine histone H1 (Upstate Biotechnology, 5 mg/ml), 940 μl H₂O, 35 μCi γ³³P-ATP) and added to 96 well plates along with 5 μl of various dilutions of the test compound in DMSO (up to 2.5%). The reaction is allowed to proceed for 5 hours before being stopped with an excess of ortho-phosphoric acid (30 μl at 2%).

γ³³P-ATP which remains unincorporated into the histone H1 is separated from phosphorylated histone H1 on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 25 μl of Microscint 20 scintillant is added, and then counted on a Packard Topcount for 30 seconds.

The % inhibition of the CDK2 activity is calculated and plotted in order to determine the concentration of test compound required to inhibit 50% of the CDK2 activity (IC₅₀).

The compounds of Examples 3 to 19, 21 to 76, 78, 80, 81 and 84 to 87 each have IC₅₀ values of less than 100 μM or provide at least 50% inhibition of the CDK2 activity at a concentration of 50 μM.

Pharmaceutical Formulations

### Example 89

(i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

(ii) Capsule Formulation

A capsule formulation is prepared by mixing 100 mg of a compound of the formula (I) with 100 mg lactose and filling the resulting mixture into standard opaque hard gelatin capsules.

### Example 90

Determination of Antifungal Activity

The antifungal activity of the compounds of the formula (I) is determined using the following protocol.

The compounds are tested against a panel of fungi including Candida parpsilosis, Candida tropicalis, Candida albicans-ATCC 36082 and Cryptococcus neoformans. The test organisms are maintained on Sabourahd Dextrose Agar slants at 4° C. Singlet suspensions of each organism are prepared by growing the yeast overnight at 27° C. on a rotating drum in yeast-nitrogen base broth (YNB) with amino acids (Difco, Detroit, Mich.), pH 7.0 with 0.05 morpholine propanesulphonic acid (MOPS). The suspension is then centrifuged and washed twice with 0.85% NaCl before sonicating the washed cell suspension for 4 seconds (Branson Sonifier, model 350, Danbury, Conn.). The singlet blastospores are counted in a haemocytometer and adjusted to the desired concentration in 0.85% NaCl.

The activity of the test compounds is determined using a modification of a broth microdilution technique. Test compounds are diluted in DMSO to a 1.0 mg/ml ratio then diluted to 64 μg/ml in YNB broth, pH 7.0 with MOPS (Fluconazole is used as the control) to provide a working solution of each compound. Using a 96-well plate, wells 1 and 3 through 12 are prepared with YNB broth, ten fold dilutions of the compound solution are made in wells 2 to 11 (concentration ranges are 64 to 0.125 μg/ml). Well 1 serves as a sterility control and blank for the spectrophotometric assays. Well 12 serves as a growth control. The microtitre plates are inoculated with 10 μl in each of well 2 to 11 (final inoculum size is 10⁴ organisms/ml). Inoculated plates are incubated for 48 hours at 35° C. The MIC values are determined spectrophotometrically by measuring the absorbance at 420 nm (Automatic Microplate Reader, DuPont Instruments, Wilmington, Del.) after agitation of the plates for 2 minutes with a vortex-mixer (Vorte-Genie 2 Mixer, Scientific Industries, Inc., Bolemia, N.Y.). The MIC endpoint is defined as the lowest drug concentration exhibiting approximately 50% (or more) reduction of the growth compared with the control well. With the turbidity assay this is defined as the lowest drug concentration at which turbidity in the well is <50% of the control (IC50). Minimal Cytolytic Concentrations (MCC) are determined by sub-culturing all wells from the 96-well plate onto a Sabourahd Dextrose Agar (SDA) plate, incubating for 1 to 2 days at 35° C. and then checking viability.

### Example 89

Protocol for the Biological Evaluation of Control of In Vivo Whole Plant Fungal Infection

Compounds of the formula (I) are dissolved in acetone, with subsequent serial dilutions in acetone to obtain a range of desired concentrations. Final treatment volumes are obtained by adding 9 volumes of 0.05% aqueous Tween-20™ or 0.01% Triton X-100™, depending upon the pathogen.

The compositions are then used to test the activity of the compounds of the invention against tomato blight (Phytophthora infestans) using the following protocol. Tomatoes (cultivar Rutgers) are grown from seed in a soil-less peat-based potting mixture until the seedlings are 10-20 cm tall. The plants are then sprayed to run-off with the test compound at a rate of 100 ppm. After 24 hours the test plants are inoculated by spraying with an aqueous sporangia suspension of Phytophthora infestans, and kept in a dew chamber overnight. The plants are then transferred to the greenhouse until disease develops on the untreated control plants.

Similar protocols are also used to test the activity of the compounds of the invention in combatting Brown Rust of Wheat (Puccinia), Powdery Mildew of Wheat (Ervsiphe vraminis), Wheat (cultivar Monon), Leaf Blotch of Wheat (Septoria tritici), and Glume Blotch of Wheat (Leptosphaeria nodorum).

Equivalents

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-79. (canceled)

80. A method for the prophylaxis or treatment of a disease state or condition mediated by a cyclin dependent kinase, which method comprises administering to a subject in need thereof a compound of the formula (I): 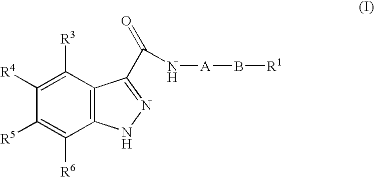
or a salt, solvate or N-oxide thereof;
wherein
A is a group R² or CH₂—R² where R² is a carbocyclic or heterocyclic group having from 3 to 12 ring members;
B is a bond or an acyclic linker group having a linking chain length of up to 3 atoms selected from C, N, S and O;
R¹ is hydrogen or a group selected from SO₂R^{b}, SO₂NR⁷R⁸, CONR⁷R⁸, NR⁷R⁹ and carbocyclic and heterocyclic groups having from 3 to 7 ring members;
R³, R⁴, R⁵ and R⁶ are the same or different and are each selected from hydrogen, halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and
R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is hydrogen or C₁₋₄ hydrocarbyl;
X¹ is O, S or NR^{c} and X² is ═O, ═S or ═NR^{c};
R⁷ is selected from hydrogen and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R⁸ is selected from R⁷ and carbocyclic and heterocyclic groups having from 3 to 12 ring members;
R⁹ is selected from R⁸, COR⁸ and SO₂R⁸;
or NR⁷R⁸ or NR⁷R⁹ may each form a heterocyclic group having from 5 to 12 ring members;
but excluding the compounds N-[(morpholin-4-yl)phenyl-1H-indazole-3-carboxamide and N-[4-(acetylaminosulphonyl)phenyl-1H-indazole-3-carboxamide.

81. A compound of the formula (II): 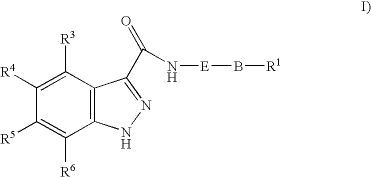
or a salt, solvate or N-oxide thereof, wherein
E is a group R¹² or CH₂—R^{12a} where R¹² is a substituted or unsubstituted, non-bridged, carbocyclic or heterocyclic group having from 3 to 12 ring members, other than a diazacycloalkyl moiety, and R^{12a} is an unsubstituted or substituted aryl or heteroaryl group having from 5 to 12 ring members;
B is a bond or an acyclic linker group having a linking chain length of up to 3 atoms selected from C, N, S and O;
R¹ is hydrogen or a group selected from SO₂R^{b}, SO₂NR⁷R⁸, CONR⁷R⁸, NR⁷R⁹ and carbocyclic and heterocyclic groups having from 3 to 7 ring members;
R³, R⁴, R⁵ and R⁶ are the same or different and are each selected from hydrogen, halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is hydrogen or C₁₋₄ hydrocarbyl;
X¹ is O, S or NR^{c} and X² is ═O, ═S or ═NR^{c};
R⁷ is selected from hydrogen and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R⁸ is selected from R⁷ and carbocyclic and heterocyclic groups having from 3 to 12 ring members;
R⁹ is selected from R⁸, COR⁸ and SO₂R⁸;
or NR⁷R⁸ or NR⁷R⁹ may each form a heterocyclic group having from 5 to 12 ring members;
and the optional substituents for the groups R¹² and R^{12a} can be one or more substituent groups R¹⁰ selected from halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is hydrogen or C₁₋₄ hydrocarbyl;
X¹ is O, S or NR^{c} and X² is ═O, ═S or ═NR^{c};
with the provisos that:
(a) when R¹² is an azacycloalkyl or diazacycloalkyl group, at least one nitrogen atom of the azacycloalkyl or diazacycloalkyl group is substituted by an acyl, sulphinyl or sulphonyl group;
(b) when E is a substituted phenyl group, the or each substituent is other than a 5-7 membered non-aromatic ring (such as cyclohexyl) having attached thereto a diazacycloalkyl moiety (such as piperazine), a nitrogen atom of which moiety bears an aryl or heteroaryl substituent; and
(c) R¹² and R^{12a} are each other than a substituted or unsubstituted imidazole moiety; but excluding the following:
(i) N-[(morpholin-4-yl)phenyl-1H-indazole-3-carboxamide;
(ii) N-[4-(acetylaminosulphonyl)phenyl-1H-indazole-3-carboxamide;
(iii) compounds wherein E is phenyl, R¹ is NR⁷R⁸ and B is a group —CH(CH₂OH)CH₂—;
(iv) compounds wherein R³ and R⁶ are both hydrogen and R⁴ and R⁵ are both methoxy;
(v) compounds wherein R³ to R⁶ are all hydrogen, wherein E is unsubstituted pyridyl or pyridylmethyl, B is a bond and R¹ is hydrogen;
(vi) compounds wherein E is phenyl substituted with one or more of alkyl, alkoxy, alkylsulphanyl, alkylsulphinyl other than meta-alkylsulphinyl, alkylsulphonyl other than meta-alkylsulphonyl, halogen, nitro and trihalomethyl, B is a bond, and R¹ is hydrogen;
(vii) compounds wherein E is a thiophene group bearing a 3-aminocarbonyl substituent;
(viii) the compound wherein E is unsubstituted phenyl or para-methoxyphenyl, and each of R³ to R⁶ is hydrogen;
(ix) N-4-methylbenzyl-1H-indazole-3-carboxamide;
(x) compounds wherein R³, R⁵ and R⁶ are each hydrogen, R⁴ is methyl and A is unsubstituted benzyl, unsubstituted phenyl, methylphenyl, meta-trifluoromethylphenyl, and ortho-methoxyphenyl;
(xi) compounds in which E is a 2,2-dimethyl-1,3-dioxane ring;
(xii) compounds containing a benzene ring substituted by a pair of meta-oriented carboxamido moieties;
(xiii) compounds wherein E is a trisubstituted phenyl group and two of the substitutents are fluoro and chloro respectively.

82. A compound according to claim 81 having the formula (III): 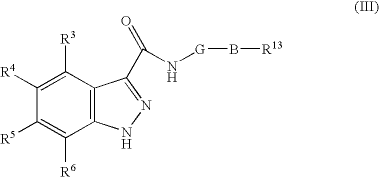
or a salt, solvate or N-oxide thereof, wherein
G is a group R¹⁴ or CH₂—R¹⁴ where R¹⁴ is a carbocyclic group having from 3 to 12 ring members;
B is a bond or an acyclic linker group having a linking chain length of up to 3 atoms selected from C, N, S and O;
R¹³ is a group selected from SO₂NR⁷R⁸, CONR⁷R⁸, NR⁷R⁹ and carbocyclic and heterocyclic groups having from 3 to 7 ring members;
R³, R⁴, R⁵ and R⁶ are the same or different and are each selected from hydrogen, halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is hydrogen or C₁₋₄ hydrocarbyl;
X¹ is O, S or NR^{c} and X² is ═O, ═S or ═NR^{c};
R⁷ is selected from hydrogen and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R⁸ is selected from R⁷ and carbocyclic and heterocyclic groups having from 3 to 12 ring members;
R⁹ is selected from R⁸, COR⁸ and SO₂R⁸;
or NR⁷R⁸ or NR⁷R⁹ may each form a heterocyclic group having from 5 to 12 ring members;
but excluding the compounds N-[(morpholin-4-yl)phenyl-1H-indazole-3-carboxamide and N-[4-(acetylaminosulphonyl)phenyl-1H-indazole-3-carboxamide; and further excluding;
(i) compounds wherein A is phenyl, R¹ is NR⁷R⁸ and B is a group —CH(CH₂OH)CH₂—;
(ii) compounds wherein R³ and R⁶ are both hydrogen and R⁴ and R⁵ are both methoxy.

83. A compound according to claim 82 having the formula (IV): 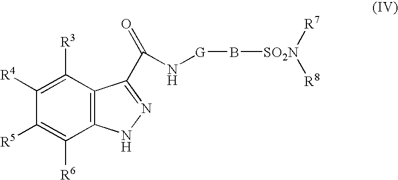
or a salt, solvate or N-oxide thereof.

84. A compound according to claim 82 having the formula (V): 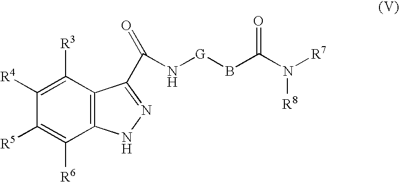
or a salt, solvate or N-oxide thereof.

85. A compound according to claim 82 having the formula (VI): 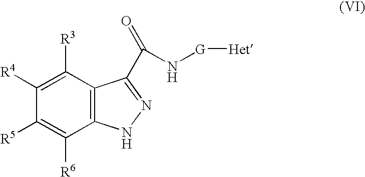
or a salt, solvate or N-oxide thereof, wherein Het′ is a heterocylic group having from 3 to 7 ring members, but excluding the compound N-[(morpholin-4-yl)phenyl]-1H-indazole-3-carboxamide.

86. A compound according to claim 82 having the formula (VII): 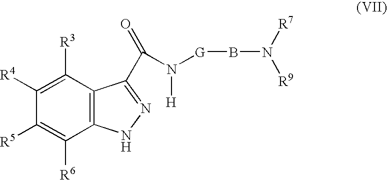
or a salt, solvate or N-oxide thereof.

87. A compound according to claim 81 having the formula (VIII): 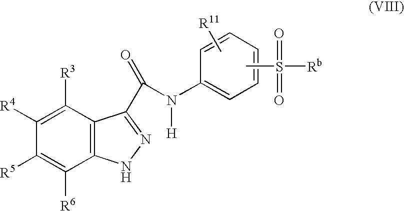
or a salt, solvate or N-oxide thereof, wherein R¹¹ represents hydrogen or one or more substituents selected from halogen, C₁₋₄ alkyl, C₁₋₄ alkoxy, trifluoromethyl and trifluoromethoxy.

88. A compound according to claim 81 having the formula (IX): 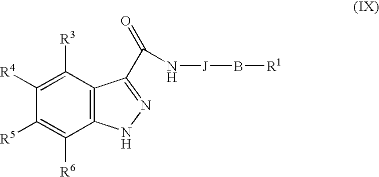
or a salt, solvate or N-oxide thereof, wherein
J is a group R¹⁵ or CH₂—R^{15a} where R¹⁵ is a substituted or unsubstituted, non-bridged heterocyclic group having from 5 to 12 ring members, other than a diazacycloalkyl moiety, and R^{15a} is an unsubstituted or substituted aryl or heteroaryl group having from 5 to 12 ring members;
B is a bond or an acyclic linker group having a linking chain length of up to 3 atoms selected from C, N, S and 0;
R¹ is hydrogen when R^{15a} is aryl or, when R^{15a} is other than aryl, R¹ is hydrogen or a group selected from SO₂R^{b}, SO₂NR⁷R⁸, CONR⁷R⁸, NR⁷R⁹ and carbocyclic and heterocyclic groups having from 3 to 7 ring members;
R³, R⁴, R⁵ and R⁶ are the same or different and are each selected from hydrogen, halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is hydrogen or C₁₋₄ hydrocarbyl;
X¹ is O, S or NR^{c} and X² is ═O, ═S or ═NR^{c};
R⁷ is selected from hydrogen and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R⁸ is selected from R⁷ and carbocyclic and heterocyclic groups having from 3 to 12 ring members;
R⁹ is selected from R⁸, COR⁸ and SO₂R⁸;
or NR⁷R⁸ or NR⁷R⁹ may each form a heterocyclic group having from 5 to 12 ring members;
and the optional substituents for the groups R¹⁵ and R^{15a} can be one or more substituent groups R¹⁰ selected from halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
provided that when R^{15a} is aryl it is not substituted either directly, or via an acyclic linker group having a linking chain length of up to 3 atoms selected from C, N, S and O, by a group selected from SO₂R^{b}, SO₂NR⁷R⁸, CONR⁷R⁸, NR⁷R⁹ and carbocyclic and heterocyclic groups having from 3 to 7 ring members;
R^{c} is hydrogen or C₁₋₄ hydrocarbyl;
X¹ is O, S or NR^{c} and X² is ═O, ═S or ═NR^{c};
with the provisos that:
(a) when R¹⁵ is an azacycloalkyl group and all of R³ to R⁶ are hydrogen, at least one nitrogen atom of the azacycloalkyl group is substituted by an acyl, sulphinyl or sulphonyl group;
(b) R¹⁵ and R^{15a} are each other than a substituted or unsubstituted imidazole moiety; but excluding the following:
(i) compounds wherein R³ and R⁶ are both hydrogen and R⁴ and R⁵ are both methoxy;
(ii) compounds wherein R³ to R⁶ are all hydrogen, J is unsubstituted pyridyl or pyridylmethyl, B is a bond and R¹ is hydrogen;
(iii) compounds wherein J is phenyl substituted with one or more of alkyl, alkoxy, alkylsulphanyl, alkylsulphinyl other than meta-alkylsulphinyl, alkylsulphonyl other than meta-alkylsulphonyl, halogen, nitro and trihalomethyl, B is a bond, and R¹ is hydrogen;
(iv) compounds wherein J is a thiophene group bearing a 3-aminocarbonyl substituent;
(v) the compound wherein J is unsubstituted phenyl or para-methoxyphenyl, and each of R³ to R⁶ is hydrogen;
(vi) N-4-methylbenzyl-1H-indazole-3-carboxamide;
(vii) compounds wherein R³, R⁵ and R⁶ are each hydrogen, R⁴ is methyl and A is unsubstituted benzyl, unsubstituted phenyl, methylphenyl, meta-trifluoromethylphenyl, and ortho-methoxyphenyl;
(viii) compounds in which J is a 2,2-dimethyl-1,3-dioxane ring;
(ix) compounds containing a benzene ring substituted by a pair of meta-oriented carboxamido moieties; and
(x) compounds wherein J is a trisubstituted phenyl group and two of the substituents are fluoro and chloro respectively.

89. A compound according to claim 81 having the formula (X): 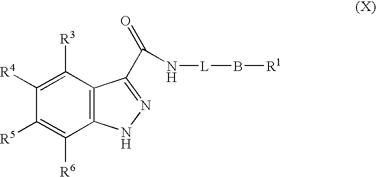
or a salt, solvate or N-oxide thereof, wherein
L is a group R¹⁶ or CH₂—R¹⁶ where R¹⁶ is a substituted or unsubstituted heteroaryl group other than imidazole, the heteroaryl group having from 5 to 12 ring members, at least one of which is nitrogen;
B is a bond or an acyclic linker group having a linking chain length of up to 3 atoms selected from C, N, S and O;
R¹ is hydrogen or a group selected from SO₂R^{b}, SO₂NR⁷R⁸, CONR⁷R⁸, NR⁷R⁹ and carbocyclic and heterocyclic groups having from 3 to 7 ring members;
R³, R⁴, R⁵ and R⁶ are the same or different and are each selected from hydrogen, halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹, provided that R⁴ and R⁵ cannot both be methoxy;
R^{c} is hydrogen or C₁₋₄ hydrocarbyl;
X¹ is O, S or NR^{c} and X² is ═O, ═S or ═NR^{c};
R⁷ is selected from hydrogen and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R⁸ is selected from R⁷ and carbocyclic and heterocyclic groups having from 3 to 12 ring members;
R⁹ is selected from R⁸, COR⁸ and SO₂R⁸;
or NR⁷R⁸ or NR⁷R⁹ may each form a heterocyclic group having from 5 to 12 ring members;
and the optional substituents for R¹⁶ can be one or more substituent groups R¹⁰ selected from halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is
selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is hydrogen or C₁₋₄ hydrocarbyl;
X¹ is O, S or NR^{c} and X² is ═O, ═S or ═NR^{c};
but excluding compounds wherein all of R³ to R⁶ are hydrogen and L-B—R¹ defines an unsubstituted pyridyl or pyridylmethyl group.

90. A compound according to claim 89 having the formula (XI): 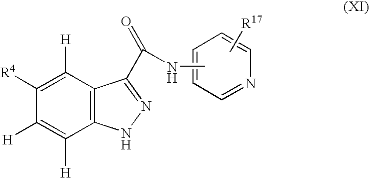
or a salt, solvate or N-oxide thereof, in which R¹⁷ is hydrogen, B—R¹ or R¹⁰, provided that at least one of R⁴ and R¹⁷ is other than hydrogen.

91. A compound according to claim 90 having the formula (XII):

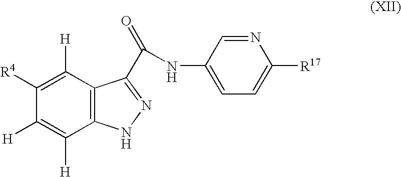

92. A compound according to claim 89 having the formula (XIII): 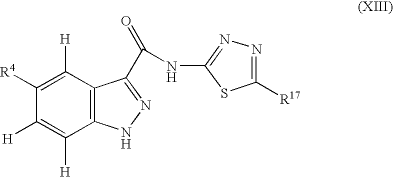
in which R¹⁷ is hydrogen, B—R¹ or R¹⁰.

93. A compound according to claim 89 having the formula (XIV): 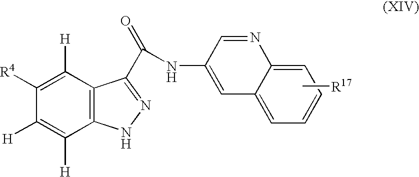
in which R¹⁷ is hydrogen, B—R¹ or R¹⁰.

94. A compound according to claim 81 having the formula (XV): 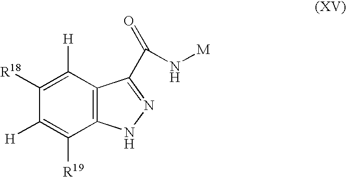
or a salt, solvate or N-oxide thereof, wherein
M is a group R²⁰ or CH₂—R²⁰ where R²⁰ is an aryl group having from 6 to 12 ring members and being optionally substituted by one or two substituent groups R¹⁰ which may be the same or different;
R¹⁸ is selected from hydrogen, halogen, and carbocyclic and heterocyclic groups having from 3 to 12 ring members;
R¹⁹ is selected from hydrogen and amino, provided that at least one of R¹⁸ and R¹⁹ is other than hydrogen;
R¹⁰ is selected from halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; provided that the aryl group R²⁰ is not substituted either directly, or via an acyclic linker group having a linking chain length of up to 3 atoms selected from C, N, S and O, by a group selected from SO₂R^{b}, SO₂NR⁷R⁸, CONR⁷R⁸, NR⁷R⁹ and carbocyclic and heterocyclic groups having from 3 to 7 ring members;
R^{c} is hydrogen or C₁₋₄ hydrocarbyl;
X¹ is O, S or NR^{c} and X² is ═O, ═S or ═NR^{c}.

95. A compound according to claim 81 having the formula (XVI): 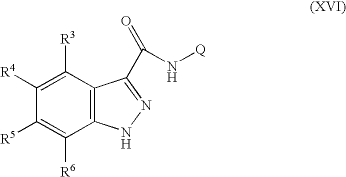
or a salt, solvate or N-oxide thereof, wherein
Q is an optionally substituted non-bridged non-aromatic heterocyclic group having from 5 to 7 ring members of which at least one is a nitrogen atom, the group being other than a diazacycloalkyl group;
R³, R⁴, R⁵ and R⁶ are the same or different and are each selected from hydrogen, halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is hydrogen or C₁₋₄ hydrocarbyl;
X¹ is O, S or NR^{c} and X² is ═O, ═S or ═NR^{c};
R⁷ is selected from hydrogen and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X;
R⁸ is selected from R⁷ and carbocyclic and heterocyclic groups having from 3 to 12 ring members;
R⁹ is selected from R⁸, COR⁸ and SO₂R⁸;
or NR⁷R⁸ or NR⁷R⁹ may each form a heterocyclic group having from 5 to 12 ring members;
and the optional substituents for the group Q can be one or more (preferably up to 2, for example 1) substituent groups R²¹ selected from SO₂R^{b}, SO₂NR⁷R⁸, CONR⁷R⁸, NR⁷R⁹, halogen, hydroxy, trifluoromethyl, cyano, nitro, carboxy, amino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is hydrogen or C₁₋₄ hydrocarbyl;
X¹ is O, S or NR^{c} and X² is ═O, ═S or ═NR^{c};
provided that when Q is an azacycloalkyl group and R³ to R⁶ are all hydrogen, at least one nitrogen atom of the azacycloalkyl or diazacycloalkyl group is substituted by an acyl, sulphinyl or sulphonyl group.

96. A compound according to claim 81 selected from:
1H-Indazole-3-carboxylic acid (4-methylsulphamoylmethyl-phenyl)-amide;
1H-Indazole-3-carboxylic acid [3-(1H-tetrazol-5-yl)-phenyl]-amide;
1H-Indazole-3-carboxylic acid [4-(acetylamino-methyl)-phenyl]-amide;
1H-Indazole-3-carboxylic acid [4-(2-oxo-pyrrolidin-1-yl)-phenyl]-amide;
1H-Indazole-3-carboxylic acid (3-oxazol-5-yl-phenyl)-amide;
1H-Indazole-3-carboxylic acid [4-(1H-imidazol-4-yl)-phenyl]-amide;
1H-Indazole-3-carboxylic acid (3-methanesulphonyl-phenyl)-amide;
1H-Indazole-3-carboxylic acid [4-(morpholine-4-sulphonyl)-phenyl]-amide;
5-Iodo-1H-indazole-3-carboxylic acid (4-sulphamoyl-phenyl)-amide;
5-Iodo-1H-indazole-3-carboxylic acid (4-methylsulphamoylmethyl-phenyl)-amide;
5-Iodo-1H-indazole-3-carboxylic acid (3-methanesulphonyl-phenyl)-amide;
5-Iodo-1H-indazole-3-carboxylic acid [4-(acetylamino-methyl)-phenyl]-amide;
5-nitro-1H-indazole-3-carboxylic acid (4-sulphamoyl-phenyl)-amide;
5-nitro-1H-indazole-3-carboxylic acid (4-methylsulphamoylmethyl-phenyl)-amide;
5-thiophen-2-yl-1H-indazole-3-carboxylic acid (4-methylsulphamoylmethyl-phenyl)-amide;
5-(3,5-dimethyl-isoxazol-4-yl)-1H-indazole-3-carboxylic acid (4-methylsulphamoylmethyl-phenyl)-amide;
5-furan-2-yl-1H-indazole-3-carboxylic acid (4-methylsulphamoylmethyl-phenyl)-amide; and
5-benzofuran-2-yl-1H-indazole-3-carboxylic acid (4-methylsulphamoylmethyl-phenyl)-amide;
N-phenyl-5-iodo-1H-indazole-3-carboxamide;
5-morpholin-4-yl-1H-indazole-3-carboxylic acid phenylamide;
5-chloro-1H-indazole-3-carboxylic acid (5-nitro-pyridin-2-yl)-amide;
1H-indazole-3-carboxylic acid (4-sulphamoyl-phenyl)-amide;
5-thiophen-2-yl-1H-indazole-3-carboxylic acid (4-methylsulphamoylmethyl-phenyl)-amide;
5-thiazol-2-yl-1H-indazole-3-carboxylic acid (4-methylsulphamoylmethyl-phenyl)-amide;
4-[(5-iodo-1H-indazole-3-carbonyl)-amino]-piperidine-1-carboxylic acid ethyl ester;
1H-indazole-3-carboxylic acid [4-(thiazol-2-ylsulphamoyl)-phenyl]-amide;
5-phenyl-1H-indazole-3-carboxylic acid (4-methylsulphamoylmethyl-phenyl)-amide;
-nitro-1H-indazole-3-carboxylic acid [4-(methanesulphonylamino-methyl)-phenyl]-amide;
4-[(5-nitro-1H-indazole-3-carbonyl)-amino]-piperidine-1-carboxylic acid ethyl ester;
5-chloro-1H-indazole-3-carboxylic acid (1-benzyl-pyrrolidin-3-yl)-amide;
4-[(5-chloro-1H-indazole-3-carbonyl)-amino]-piperidine-1-carboxylic acid ethyl ester;
5-iodo-1H-indazole-3-carboxylic acid (6-methoxy-pyridin-3-yl)-amide;
5-iodo-1H-indazole-3-carboxylic acid pyridin-3-yl-amide;
5-iodo-1H-indazole-3-carboxylic acid quinolin-3-ylamide;
5-iodo-1H-indazole-3-carboxylic acid (tetrahydro-pyran-4-yl)-amide;
5-chloro-1H-indazole-3-carboxylic acid (1-methyl-piperidin-4-yl)-amide;
5-iodo-1H-indazole-3-carboxylic acid (2-chloro-pyridin-3-yl)-amide;
5-chloro-1H-indazole-3-carboxylic acid benzylamide;
5-chloro-1H-indazole-3-carboxylic acid 4-(4-methyl-piperazin-1-yl)-benzylamide;
5-chloro-1H-indazole-3-carboxylic acid pyridin-3-ylamide;
5-iodo-1H-indazole-3-carboxylic acid (6-cyano-pyridin-3-yl)-amide;
5-chloro-1H-indazole-3-carboxylic acid phenylamide;
5-iodo-1H-indazole-3-carboxylic acid (6-methyl-pyridazin-3-yl)-amide;
5-chloro-1H-indazole-3-carboxylic acid (5-ethyl-[1,3,4]thiadiazol-2-yl)-amide;
5-iodo-1H-indazole-3-carboxylic acid (4-morpholin-4-yl-phenyl)-amide;
5-iodo-1H-indazole-3-carboxylic acid (2-oxo-1,2-dihydro-pyridin-3-yl)-amide;
1H-indazole-3-carboxylic acid (4-morpholin-4-yl-phenyl)-amide;
5-nitro-1H-indazole-3-carboxylic acid phenylamide;
5-iodo-1H-indazole-3-carboxylic acid (6-chloro-pyridin-3-yl)-amide;
4-[(1H-indazole-3-carbonyl)-amino]-piperidine-1-carboxylic acid tert-butyl ester;
5-iodo-1H-indazole-3-carboxylic acid (4-fluoro-phenyl)-amide;
5-iodo-1H-indazole-3-carboxylic acid (6-acetylamino-pyridin-3-yl)-amide;
5-amino-1H-indazole-3-carboxylic acid phenylamide;
5-iodo-1H-indazole-3-carboxylic acid (4-methylaminosulphonylmethyl-phenyl)-amide;
5-amino-1H-indazole-3-carboxylic acid (4-sulphamoyl-phenyl)-amide;
7-amino-1H-indazole-3-carboxylic acid (4-sulphamoyl-phenyl)-amide;
5-[3-(2-chloro-ethyl)-ureido]-1H-indazole-3-carboxylic acid (4-methylsulphamoyl-methyl-phenyl)-amide;
5-nitro-1H-indazole-3-carboxylic acid (4-methylsulphamoylmethyl-phenyl)-amide;
5-amino-1H-indazole-3-carboxylic acid (4-methylsulphamoylmethyl-phenyl)-amide;
5-iodo-1H-indazole-3-carboxylic acid piperidin-4-ylamide
5-chloro-1H-indazole-3-carboxylic acid [4-(acetylamino-methyl)-phenyl]-amide;
1H-indazole-3-carboxylic acid [1-(2,2,2 trifluoro-acetyl)-Piperidin-4-yl]-amide;
1H-indazole-3-carboxylic acid piperidin-4-ylamide;
1H-indazole-3-carboxylic acid (1-acetyl-piperidin-4-yl)-amide;
1H-indazole-3-carboxylic acid (1-methanesulphonyl-piperidin-4-yl)-amide;
1H-indazole-3-carboxylic acid (4-fluoro-phenyl)-amide;
4-bromo-1H-indazole-3-carboxylic acid (4-fluoro-phenyl)-amide;
5-nitro-1H-indazole-3-carboxylic acid (4-fluorophenyl)-amide;
5-amino-1H-indazole-3-carboxylic acid (4-fluorophenyl)-amide;
5-amino-4-bromo-1H-indazole-3-carboxylic acid (4-fluorophenyl)-amide;
5-methyl-1H-indazole-3-carboxylic acid (4-fluoro-phenyl)-amide;
6-bromo-1H-indazole-3-carboxylic acid (4-fluoro-phenyl)-amide;
5-chloro-1H-indazole-3-carboxylic acid (4-morpholin-4-yl-phenyl)-amide;
5-chloro-1H-indazole-3-carboxylic acid [3-(1H-tetrazol-5-yl)-phenyl]-amide;
5-iodo-1H-indazole-3-carboxylic acid (4-pyrrolidin-1-ylmethyl-phenyl)-amide;
5-chloro-1H-indazole-3-carboxylic acid [4-(thiazol-2-ylsulphamoyl)-phenyl]-amide;
5-chloro-1H-indazole-3-carboxylic acid (4-fluoro-phenyl)-amide;
3-[(5-chloro-1H-indazole-3-carbonyl)-amino]-pyrrolidine-1-carboxylic acid methyl ester;
5-fluoro-1H-indazole-3-carboxylic acid phenylamide;
5-morpholin-4-yl-1H-indazole-3-carboxylic acid (6-chloro-pyridin-3-yl)-amide;
1H-indazole-3-carboxylic acid (6-chloro-pyridin-3-yl)-amide;
5-phenethyl-1H-indazole-3-carboxylic acid phenylamide;
5-(1,1-dioxo-1 lambda*6*-isothiazolidin-2-yl)-1H-indazole-3-carboxylic acid phenylamide;
5-biphenyl-2-yl-1H-indazole-3-carboxylic acid phenylamide;
5-pyrrolidin-1-yl-1H-indazole-3-carboxylic acid phenylamide;
5-chloro-1H-indazole-3-carboxylic acid [5-(tetrahydro-furan-2-yl)-[1,3,4]thiadiazol-2-yl]-amide; and
5-nitro-1H-indazole-3-carboxylic acid (3-methanesulphonyl-phenyl)-amide.

97. A pharmaceutical composition comprising a compound as defined in claim 81 and a pharmaceutically acceptable carrier.
